(19) United States
(12) Patent Application Publication
(10) Pub. No.: US 2006/0223117 A1
(43) Pub. Date: Oct. 5, 2006

(54) Novel splice variants of human epithelial sodium channel genes expressed in human taste tissue and uses thereof

(75) Inventors: Bryan Moyer, San Diego, CA (US); Fernando Echeverri, Chula Vista, CA (US); Min Lu, San Diego, CA (US); Bianca Laita, Oceanside, CA (US)

Correspondence Address: DUANE MORRIS LLP, 1667 K. STREET, N.W., SUITE 700, WASHINGTON, DC, 20006-1608, US

(73) Assignee: SENOMYX, INC., LaJolla, CA (US)

(21) Appl. No.: 11/345,403

(22) Filed: Feb. 2, 2006

Related U.S. Application Data
(60) Provisional application No. 60/675,719, filed on Apr. 29, 2005.
(60) Provisional application No. 60/650,116, filed on Feb. 7, 2005.

(51) Int. Cl. G01N 33/53; C07H 21/04; C07K 14/705; C07K 16/28; C12P 21/06

(52) U.S. Cl. 435/7.1; 530/350; 530/388.22; 435/320.1; 435/325; 536/23.5; 435/69.1

(57) ABSTRACT

Nucleic acid sequences encoding novel splice variants that encode subunits of an ENaC expressed in human taste tissue are provided. These splice variants when expressed in association with other ENaC subunits, i.e., α, β and γ subunits or α, β and Δ subunits may be used to produce amiloride-insensitive ENACs. The resultant amiloride-insensitive ENaCs are useful in in vitro assays for identifying ENaC modulators that modulate taste (enhance or inhibit), particularly human salty taste.

### CROSS REFERENCE TO RELATED APPLICATIONS

This provisional patent application related to U.S. Provisional Application Ser. No. 60/287,413 filed May 1, 2001, U.S. Ser. No. 10/133,573 filed Apr. 29, 2002, and Provisional Application U.S. 60/650,116 filed Feb. 7, 2005, all of which are incorporated by reference in their entireties herein.

### FIELD OF THE INVENTION

The present invention relates to the discovery of novel splice variants for human epithelial sodium channel genes, including α, β, and γ channel subunits expressed in human taste tissues. The present invention further relates to the expression of these splice variants alone or in association with other human epithelial channel genes and variants to produce functional amiloride-insensitive sodium channels and the use of these sodium channels in assays to profile, screen for and identify taste (salty taste) modulating compounds. Preferably, these assays will comprise high throughput cell-based assays that use mammalian cells which express a sodium channel comprising one or more splice variant genes according to the present invention.

### BACKGROUND OF THE INVENTION

An amiloride-sensitive epithelial sodium channel (ENaC) mediates sodium influx across the apical membrane of taste buds cells in the tongue (Heck, et al, Science (1984) 223: 403-405). ENaC, a member of the ENaC/degenerin superfamily of ion channels involved in sodium transport, is composed of three partially homologous α, β, and γ subunits expressed at both the RNA and protein level in fungiform, foliate, and circumvallate papilla as well as the lingual epithelium in taste tissue (Li, et al, Proc. Natl. Acad. Sci. (1994) 91: 1814-1818; Kretz, et al, J. Histochem. Cytochem. (1999) 47(1): 51-64; Lin, et al, J. Comp. Neurol. (1999) 405: 406-420; Xiao-Jiang, et al, Mol. Pharmacol. (1995) 47: 1133-1140).

Complementary DNAs (cDNAs) encoding an amiloride-sensitive epithelial sodium channel (ENaC) have previously been isolated from kidney cells and expressed in a mammalian cell line. The channel expressed in this system has been shown to have similar properties to the distal renal sodium channel, i.e., high sodium selectivity, low conductance, and amiloride sensitivity. One form of the naturally occurring ENaC channel is comprised of three subunits of similar structure: alpha (OMIM Entry 600228), beta (OMIM Entry 600760), and gamma (OMIM Entry 600761). Each of the subunits is predicted to contain 2 transmembrane spanning domains, intracellular amino- and carboxy-termini, and a cysteine-rich extracellular domain. The three subunits share 32 to 37% identity in amino acid sequence.

Some alternatively spliced forms of alpha-ENaC have previously been identified, indicating heterogeneity of alpha subunits of amiloride-sensitive sodium channels that may account for the multiple species of proteins observed during purification of the channel (U.S. Pat. No. 5,693,756, which is herein incorporated by reference). Further, based on published electrophysiological data and the discovery that ENaC occurs in taste bud cells, a model of salty taste transduction mediated by ENaC has been constructed. As such, the use of ENaC in the identification of substances which stimulate or block salty taste perception has been suggested (U.S. Pat. No. 5,693,756, supra). Also, the present assignee, Senomyx, recently filed a provisional application U.S. 60/650,116 which also relates to the identification of novel splice variants of human epithelial channel genes and their use to provide amiloride-insensitive ion channels.

An inhibitor of ENaC sodium channel function, amiloride, attenuates gustatory responses to sodium chloride in numerous non-mammalian as well as mammalian species, including rodents but not humans (Halpern, Neuroscience and Behavior Reviews (1998) 23: 5-47 and all references within; Liu, et al, Neuron (2003) 39: 133-146; Zhao, et al, Cell (2003) 115: 255-266). In humans, amiloride has been reported to reduce the intensity of sodium chloride by only 15-20% when used at concentrations that specifically inhibit ENaC function (Halpern, Neurosciences and Behavior Reviews (1998) 23:5-47 and all references within; Feldman, et al, J. Neurophysiol. (2003) 90(3): 2060-2064). Experiments performed at Senomyx did not demonstrate a significant effect of amiloride, or the more potent amiloride derivative phenamil, on perceived salt intensity when tested at levels 300-fold (for amiloride) and 3000-fold (for benzamil) above IC50 values in oocytes. In addition, enhancers of the kidney form of ENaC did not promote salt intensity when tested at levels 100-fold above EC50 values in oocytes. Since taste mechanisms for sweet, bitter, and savory (umami) taste are conserved between rodents and humans, it is likely that salt taste mechanisms are also similar between species. Therefore, to explain the differential effect of amiloride on salt taste between rodents and humans, we hypothesize that a splice variant(s) of ENaC exists in human taste bud cells that is not or weakly inhibited by amiloride and also not or weakly activated by our kidney ENaC enhancers. Thus, experiments were performed to identify novel ENaC splice variants in human taste tissue.

Cell-based functional expression systems commonly used for the physiological characterization of ENaC are Xenopus laevis oocytes and cultured mammalian cell lines. The oocyte system has advantages in that it allows the direct injection of multiple mRNAs, provides high levels of protein expression, and can accommodate the deleterious effects inherent in the over expression of ENaC. The drawbacks of this system are that electrophysiological recording in Xenopus oocytes is not amenable to screening large numbers of compounds and that the oocyte is not a mammalian system. Studies of the electrophysiological properties of rodent ENaC in mammalian cell lines (HEK293 and MDCK) stably expressing the channel have been reported in the literature. In these studies, channel function was assayed using electrophysiological techniques.

Recently, Senomyx developed a fluorescent-based high throughput mammalian cell based assays for profiling and screening of putative modulators of ENaC that screen mammalian cells that express a functional ENaC loaded with membrane potential fluorescent dyes or sodium-sensitive fluorescent dyes against a putative ENaC modulatory compound. These assays may be used to identify compounds that enhance or block ENaC function which potentially are useful in modulating salty taste in humans. These assays are described in U.S. Ser. No. 10/133,573 filed Apr. 29, 2002 incorporated by reference in its entirety herein. Also, as noted above, Senomyx recently filed provision application 60/650,116 also relating to novel splice variants of human epithelial channel genes, the use thereof to provide amiloride-insensitive ion channels and their use thereof in assays to identify salt taste modulators. This invention relates to the identification of another ENaC splice variant and use thereof alone and in combination with other ENaC subunits to produce amiloride-insensitive ion channels.

### SUMMARY OF THE INVENTION

Using mammalian cells which express the ENaC gene sequences disclosed in a prior application by the parent Assignee U.S. Ser. No. 10/133,573 filed Apr. 29, 2002, (incorporated by reference in its entirety herein), it was found that amiloride and the more patent amiloride derivative phenomil did not exhibit a significant effect on perceived salt intensity when tested at levels 300-fold (for amiloride) and 3000-fold (for benzamil) above IC50, levels in oocytes. (Unpublished experiments conducted by Senomyx). Additionally, enhancers of the kidney form of ENaC did not promote salt intensity when tested at levels 100-fold above EC50 values in oocytes. (unpublished experiments conducted by Senomyx).

Since taste mechanisms for sweet, bitter and savory (umami) taste are substantially conserved in rodents and humans, it is reasonable to assume that taste mechanisms which regulate salty taste are also conserved. Therefore, given the disparate effect of amiloride on salty taste between rodents and humans (obtained using cell-based assays which utilized the human kidney derived ENaC gene sequences disclosed in U.S. Ser. No. 10/133/573) it was hypothesized that this aberration may be attributable to the existence of splice variant(s) of ENaC expressed in human taste buds that are not or are weakly inhibited by amiloride and/or not or weakly inhibited by kidney ENaC enhancers. (This hypothesis is also supported by the existence of other alternatively spliced forms of the alpha subunit of ENaC reported in U.S. Pat. No. 5,693,756).

Therefore, the present invention relates to the identification of novel splice variants of human ENaC subunit genes which are expressed in human taste tissue.

Additionally, the present invention relates to the expression of the identified splice variants (α, β, and γ subunits) alone or in association with other splice variants or other known ENaC subunit sequences e.g., the human ENaC subunit sequences reported in U.S. Ser. No. 133,573, and those disclosed in U.S. Provisional 60/650,116 preferably in mammalian cells.

Also, the present invention relates to the use of functional ENaC channels comprised of one or more splice variants according to the invention in assays, preferably high throughput mammalian or amphibian cell based assays such as are disclosed in U.S. Ser. No. 133,573 for identifying compounds that modulate (enhance or inhibit) ENaC function.

Further, the present invention relates to the use of the compounds identified in these assays in human taste tests to confirm their modulatory effect on salty taste perception.

Also, the present invention relates to the use of compounds identified in such assays as food additives for modulating salty taste in salty foods and beverages.

### BRIEF DESCRIPTION OF THE FIGURES

FIG. 1 shows basal currents in Xenopus oocytes expressing combinations of specific ENaC subunit(s) (splice variant according to the invention) compared against uninjected oocytes as a control. These results indicate that α1β7 and α2βγ ENaC channels exhibit similar NMDGC1, LiCl and amiloride-sensitive currents whereas α1βγ splice variant ENaC channels do not exhibit basal activity (currents are not significantly different from uninjected oocytes).

FIG. 2 shows the potency of amiloride and another (proprietary) compound (that activates kidney EnaC) on α1βγ and α2βγ ENaC channels. The IC50 value for amiloride was 112+/−5 nM for α1βγ ENaC and 153+/−25 nM for α2βγ ENaC. The EC50 value for the proprietary compound 6363969 was 1.1+/−0.2 μM for α1βγ ENaC and 1.2+/−0.5 μM for α2βγ EnaC.

### DETAILED DESCRIPTION OF THE INVENTION

The term “salty taste” or “salty taste perception” as used herein refers to a subject's perception or response to salt taste stimuli. As discussed above, it is believed that hENaC is involved in salty taste perception in human subjects. Such stimuli include compounds such as NaCl that elicits its active ENaCs, preferably hENaC.

The terms “ENaC” subunit protein or a fragment thereof, or a nucleic acid encoding one of three subunits of “ENaC” protein or a fragment thereof refer to nucleic acids and polypeptides, polymorphic variants, alleles, mutants, and interspecies homologues that: (1) have an amino acid sequence that has greater than about 80% amino acid sequence identity, 85%, 90%, preferably 91%, 92%, 93%, 94%, 95%, 96%, 97%, 98% or 99% or greater amino acid sequence identity, preferably over a region of over a region of at least about 25, 50, 100, 200, or 500, or more amino acids, to an amino acid sequence contained in the nucleic acid sequence contained in SEQ ID NO:1, 3, 5, 7, 9, 11, 13, 15, 17, or 19, or (2) specifically bind to antibodies, e.g., polyclonal antibodies, raised against an immunogen comprising an amino acid sequence encoded by SEQ ID NO:2, 4, 6, 8, 10, 12, 14, 16, 18, 20, 22 or 24 or immunogenic fragments thereof, and conservatively modified variants thereof, or (3) specifically hybridize under stringent hybridization conditions to an anti-sense strand corresponding to a nucleic acid sequence encoding an ENaC protein, e.g., SEQ ID NO:1, 3, 5, 7, 9, 11, 13, 15, 17, or 19 or their complements, and conservatively modified variants thereof, or (4) have a nucleic acid sequence that has greater than about 80% sequence identity, 85%, 90%, preferably 91%, 92%, 93%, 94%, 95%, 96%, 97%, 98% or 99%, or higher nucleotide sequence identity, preferably over a region of at least about 25, 50, 100, 200, 500, 1000, or more nucleotides, to SEQ ID NO: 1, 3, 5, 7, 9, 11, 13, 15, 17, or 19, or their complements, or (5) is functionally equivalent to the hENaC described herein in a sodium conductance assay when expressed in a HEK cell and tested by using two electrode whole cell electrophysiology or by the change in fluorescence of a membrane potential dye in response to sodium or lithium.

Functionally equivalent ENaC proteins include ENaC subunits with primary sequences different than those identified infra, but which possess an equivalent function as determined by functional assays, e.g., sodium conductance assays as described infra. By “determining the functional effect” refers to assaying the effect of a compound that increases or decreases a parameter that is indirectly or directly under the influence of an ENaC polypeptide e.g., functional, physical and chemical effects. Such functional effects include, but are not limited to, changes in ion flux, membrane potential, current amplitude, and voltage gating, a as well as other biological effects such as changes in gene expression of any marker genes, and the like. The ion flux can include any ion that passes through the channel, e.g., sodium or lithium and analogs thereof such as radioisotopes. Such functional effects can be measured by any means known to those skilled in the art, e.g., by the use of two electrode electrophysiology or voltage-sensitive dyes, or by measuring changes in parameters such as spectroscopic characteristics (e.g., fluorescence, absorbance, refractive index), hydrodynamic (e.g., shape), chromatographic, or solubility properties. Preferably, ENaC function will be evaluated by using two electrode whole cell electrophysiology or by monitoring the change in fluorescence of a membrane potential dye in response to sodium or lithium.

“Inhibitors”, “activators”, and “modulators” of ENaC polynucleotide and polypeptide sequences are used to refer to activating, inhibitory, or modulating molecules identified using cell-based assays of ENaC polynucleotide and polypeptide sequences. Inhibitors are compounds that, e.g., bind to, partially or totally block activity, decrease, prevent, delay activation, inactivate, desensitize, or down regulate the activity or expression of ENaC proteins, e.g., antagonists. “Activators” are compounds that increase, open, activate, facilitate, enhance activation, sensitize, agonize, or up regulate ENaC protein activity. Inhibitors, activators, or modulators also include genetically modified versions of ENaC proteins, e.g., versions with altered activity, as well as naturally occurring and synthetic ligands, antagonists, agonists, peptides, cyclic peptides, nucleic acids, antibodies, antisense molecules, ribozymes, small organic molecules and the like. Such assays for inhibitors and activators include, e.g., expressing ENaC protein in cells, cell extracts, or cell membranes, applying putative modulator compounds, and then determining the functional effects on activity, as described above.

Samples or assays comprising ENaC proteins that are treated with a potential activator, inhibitor, or modulator are compared to control samples without the inhibitor, activator, or modulator to examine the extent of activation, inhibition or modulation. In one embodiment of the assay, compounds are tested for their effect on the response of cells provided with a suboptimal sodium concentration. Control cells, treated with the suboptimal concentration of sodium but lacking a compound, typically exhibit a 10-20% of the maximal response. Compounds that increase the response of the suboptimal sodium concentration above the 10-20% level are putative ENaC enhancers. In contrast, compounds that reduce the response to below 10% are putative ENaC enhancers.

The term “test compound” or “test candidate” or “modulator” or grammatical equivalents thereof as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid (e.g., a sphingolipid), fatty acid, polynucleotide, oligonucleotide, etc., to be tested for the capacity to modulate ENaC activity. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., enhancing activity, creating variants of the lead compound, and evaluating the property and activity of those variant compounds. Preferably, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” includes sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 80% identity, preferably 85%, 90%, 91%, 92%, 93%, 94%, 95%, 96%, 97%, 98%, 99%, or higher identity over a specified region (e.g., nucleotide sequences SEQ ID NO: 1, 3, 5, 7, 9, 11, 13, 15, 17, or 19), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but those functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologous, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). As noted previously, the invention embraces cells that express ENaC subunit polypeptides having primary sequences different than those disclosed in the subject application that are functionally equivalent in appropriate assays, e.g., using whole cell sodium conductance assays described in detail infra.

Macromolecular structures such as polypeptide structures can be described in terms of various levels of organization. For a general discussion of this organization, see, e.g., Alberts et al., Molecular Biology of the Cell (3.sup.rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered three-dimensional structures within a polypeptide. These structures are commonly known as domains, e.g., transmembrane domains pore domains, and cytoplasmic tail domains. Domains are portions of a polypeptide that form a compact unit of the polypeptide and are typically 15 to 350 amino acids long. Exemplary domains include extracellular domains, transmembrane domains, and cytoplasmic domains. Typical domains are made up of sections of lesser organization such as stretches of .quadrature.-sheet and .quadrature.-helices. “Tertiary structure” refers to the complete three-dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

ENaC nucleic acid sequences also include single nucleotide polymorphisms which encode ENaC subunits that are functionally equivalent to the ENaC polypeptides disclosed herein when assayed using appropriate assays, in the sodium conductance assays described herein.

Membrane potential dyes or voltage-sensitive dyes refer to a molecule or combinations of molecules that change fluorescent properties upon membrane depolarization. These dyes can be used to detect the changes in activity of an ion channel such as ENaC expressed in a cell.

A “label” or a “detectable moiety” is a composition detectable by spectroscopic, photochemical, biochemical, immunochemical, chemical, or other physical means. For example, useful labels include .sup.32P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In the present invention this typically refers to cells that have been transfected with nucleic acid sequences that encode one or more ENaC subunits.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). The term “heterologous” when used with reference to cellular expression of a gene, cDNA, mRNA or protein indicates that the gene, cDNA, mRNA, or protein is not normally expressed in the cell or is from another species than the original source of the cells.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

Particularly, such an antibody includes one which specifically binds to an ENaC disclosed herein, or a mixture of antibodies that specifically bind such ENaC polypeptides.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to ENaC subunit proteins, e.g., the ENaC alpha, beta, gamma or delta subunits as encoded by SEQ ID NO:1, 3, 5, 7, 9, 11, 13, 15, 17, or 19, polymorphic variants, alleles, orthologs, and conservatively modified variants, or splice variants, or portions thereof, can be selected to obtain only those polyclonal antibodies that are specifically immunoreactive with ENaC subunit proteins i.e., ENaC alpha, beta, gamma or delta subunits, e.g., those having the amino acid sequences contained in SEQ ID NO.: 2, 4, 6, 8, 10, 12, 14, 16, 18, and 20, and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

The present Assignee Senomyx Inc. previously developed high-throughput assays for identifying modulations of human ENaC. These high throughput assays used cells that expressed ENaC subunit sequences expressed in human kidney. These assays used human kidney derived ENaC sequences partly based on the fact that human kidney tissues are much more widely available than human taste tissues. However, because the experiments performed by Senomyx using amiloride and phenamil did not demonstrate a significant effect on perceived salt intensity, even at very high concentrations 300-fold (amiloride) and 3000-fold (benzamil) above IC50 values in oocytes), it was hypothesized that these results may be explained by the existence of splice variant(s) of human ENaC that are expressed in human taste buds, which are (analogous to rodent ENaC) not or weakly inhibited by amiloride and/or not or weakly activated by other kidney ENaC enhancers.

The present invention therefore relates to the identification, characterization, and expression of novel ENaC splice variants which are expressed in human taste tissue. The present invention further relates to the expression of such splice variants in association with other EnaC subunits to produce functional ENaCs and the use thereof in assays to identify EnaC modulators using assays disclosed infra. The methods by which the present inventors cloned and characterized these splice variants and their sequences is provided in the examples and Sequence Listing which follow.

Having generally described the invention, the same will be more readily understood by reference to the following examples, which are provided by way of illustration and are not intended as limiting. It is understood that various modification and changes can be made to the herein disclosed exemplary embodiments without departing from the spirit and scope of the invention.

### EXAMPLE 1

### Isolation and Sequencing of ENaC Splice Variants According to the Invention

Human circumvallate taste papillae on tongues were obtained from 2 independent post-mortem donors through a contract with Dr. Mark Whitehead and UCSD (contacts WHIM01-11 and CALU11-11). Total RNA was purified using the TOTALLY RNA purification kit (Ambion) and cDNA was synthesized using SuperScriptIII (Invitrogen) following the manufacturer's instructions. RT-PCR analysis confirmed expression of taste-specific genes including T1R1, T1R3, gustducin, PLB-β2, and TRPM5, demonstrating that obtained tissue actually contained taste buds. Primers spanning the full-length open reading frames for α1, α2, β, and γ ENaC were used to amplify ENaC channel subunit mRNAs. PCR products were cloned into the pGEM-T Easy vector (Promega) following the manufacturer's instructions. Between 50 and 150 total clones were analyzed (number of clones is total from both donors) by DNA sequencing to compare taste ENaC mRNA sequences to reference kidney ENaC mRNA sequences and to determine if taste ENaC clones exhibited alternative splicing. Published ENaC genomic structures, including defined exon-intron boundaries, were used to determine if taste ENaC clones include or exclude exon and intron sequences.

For αl ENaC, 69 clones were analyzed and one splice variant was observed: variant α1A (found in 1 clone). Other clones were identical to reference kidney αl ENaC sequence. In the α1Δ splice variant, nucleotides #979-1035 (amino acids # 327-345 in the extracellular loop) are deleted from the be inning of exon #6 (57 nucleotides and 19 9 amino acids) due to use of an alternative 3′ splice acceptor site. This variant does not remove sites implicated in amiloride block of ENaC function and has previously been described in scientific literature from human H441 lung epithelial cells as well as human lung and heart tissue (Tucker J K, Tamba K, Lee Y j, Shen L L, Warnock D G, Oh Y. Cloning and functional studies of splice variants of the alpha-subunit of the amiloride-sensitive Na+ channel. Am J. Physiol. 1998 April; 274(4 Pt 1):C1081-9). (This variant was also observed in human CV taste tissue from ILSbio disclosed in our previous patent application 60/650,116 incorporated by reference in its entirety herein).

For α2 ENaC, 51 clones were analyzed and one variant was observed: variant α2A (found in 1 clone). Other clones were identical to reference kidney α2 ENaC sequence. In the α2Δ splice variant, nucleotides #1157-1213 (amino acids # 386-404 in the extracellular loop) are deleted from the beginning of exon #6 (57 nucleotides and 19 amino acids) due to use of an alternative 3′ splice acceptor site. This variant does not remove sites implicated in amiloride block of ENaC function and a similar variant has previously been described for al ENaC in human H441 lung epithelial cells as well as human lung and heart tissue (Tucker J K, Tamba K, Lee Y j, Shen L L, Warnock D G, Oh Y. Cloning and functional studies of splice variants of the alpha-subunit of the amiloride-sensitive Na+ channel. Am J. Physiol. 1998 April; 274(4 Pt 1):C1081-9). Variant α2A arises from similar splicing events found in variant α1A. This variant was also observed in human CV taste tissue from ILSbio in our previous invention disclosure (2-3-05).

For β ENaC, 153 clones were analyzed and three splice variants were observed: variants PA (found in 13 clones), βB (found in 2 clones), and β* (found in 8 clones).

Other clones were identical to reference kidney β ENaC sequence. In βA splice variants, nucleotides #1045-1152 (amino acids # 349-384 in the extracellular loop) are deleted due to skipping of exon #7 (108 nucleotides and 36 amino acids). In βB splice variants, nucleotides #312-776 (amino acids # 105-259 in the extracellular loop) are deleted due to complete skipping (exclusion) of exons #3 and 4 (465 nucleotides and 155 amino acids). In β* splice variants, nucleotides #1036-1044 (amino acids # 346-348 in the extracellular loop) are deleted due use of an alternative 5′ splice site at the end of exon #6 (removal of 9 nucleotides and 3 amino acids). These β variants do not remove sites implicated in amiloride block of ENaC. βA and βB variants were also observed in human CV taste tissue from ILSbio and disclosed in our previous patent application 60/650,116. However, β* comprises a new splice variant not previously identified from Senomyx.

For γ ENaC, 94 clones were analyzed and one splice variant was observed: variant γA (found in 5 clones). Other clones were identical to reference kidney γ ENaC sequence. In γA splice variants, nucleotides #1078-1176 (amino acids # 360-392 in the extracellular loop) are deleted due to skipping of exon #7 (99 nucleotides and 33 amino acids). This γ variant does not remove sites implicated in amiloride block of ENaC. This variant was also observed in human CV taste tissue from ILSbio disclosed in our previous patent application 60/650,116.

### EXAMPLE 2

### Functional Expression of ENaC Comprising Splice Variant According to the Invention

Experiments are conducted to identify a human taste tissue expressed ENaC splice variant according to the invention which when expressed in association with other splice variants according to the invention and/or other ENaC subunit sequences (e.g., Kidney-derived ENaC subunit sequences disclosed in U.S. Ser. No. 133,573 incorporated by reference in its entirety herein) yields a functional ENaC.

In these experiments, functionality is determined based on the following properties:

1) basal sodium channel activity;

2) weak or no inhibitory effect of amiloride on basal sodium channel currents; and

3) weak or no stimulating effect on kidney ENaC enhancers on channel currents.

An ENaC channel exhibiting such properties will confirm our hypothesis concerning the reason for the lack of a detectable effect of amiloride kidney ENaC enhancers in human taste tests (using kidney-derived human ENaC subunit sequences). Moreover, compounds which modulate ENaC's that possess such properties should be functional (exhibit a modulatory effect on salty taste) in human taste tests.

### Materials and Methods Used

The following ENaC splice variant combinations were tested in Xenopus oocytes using a proprietary oocyte assay at Senomyx (OpusXpress assay™ system). This assay system is described in U.S. Ser. No. 10/133,573 incorporated by reference herein. For each test group, oocytes were injected with 1-3 mg of cRNA for each subunit and whole cell currents were measured by two-electrode voltage clamping 24-48 hours post-injection.
1) α1βγ (kidney ENaC as positive control)
2) α2βγ (α2ENaC (SEQ ID NO: 5) from human taste tissue, βγ subunits from human kidney)
3) α1Aβγ (α1A ENaC splice variant (SEQ ID NO:3) from human taste tissue, βγ from human tissue)

To test basal channel activity, currents were measured under the following conditions.

1) NMDG-C1 (-NMDG⁺ is a large action that is not permeable through ENaC and is used to determine basal sodium-dependent currents)

2) LiCl (—Li+ is a small cation that is two-fold more permeable through ENaC compared to Na+ and is used to determine basal sodium-dependent currents.)

3) Amiloride—(Amiloride is an open channel ENaC blocker used to determine basal sodium-dependent currents).

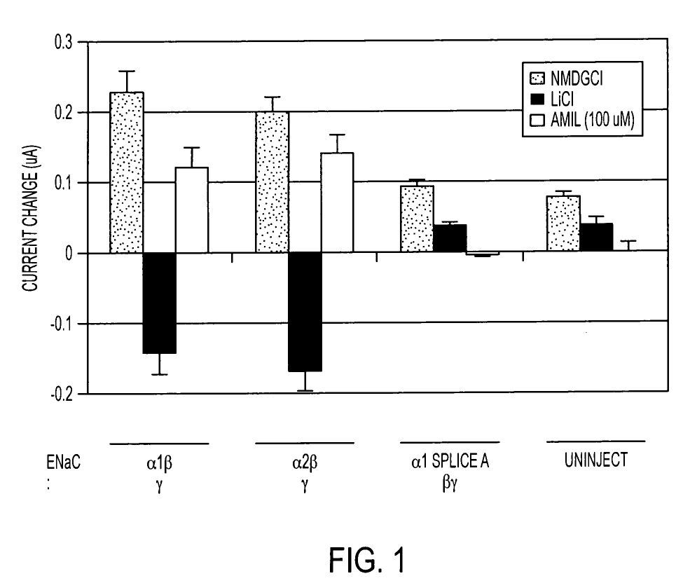

The results of these experiments are contained in FIG. 1. These results show basal currents in Xenopus oocytes expressing three different ENaC subunit combinations and uninjected oocytes as a negative control. More particularly, these results reveal that α1βγ and α2βγENaC channels exhibit similar NMDGC, LiCl and amiloride-sensitive currents. By contrast, α1Aβγ splice variant ENaC channels do not exhibit basal activity (currents are not significantly different from uninjected oocytes).

These same experiments are being conducted using all potential ENaC combinations according to the invention (cell expressing at least one α, β, γ subunit wherein each or all potentially can comprise a splice variant according to the invention). Additionally, in these experiments the γ subunit can particularly be substituted with an ENaC delta subunit sequence.

### EXAMPLE 3

### Effect of Kindey ENaC Enhancers on Channel Activity

Using oocytes which express the ENaC subunit combinations described in the previous example, the effect of various kidney ENaC enhancers was tested on channel activity. These experiments used proprietary compounds previously shown by the present Assignee to enhance kidney ENaC channel function:

1) 3912721 (A proprietary compound produced by Pictet-Spenglar Chemistry that enhances kidney ENaC);

2) 8246776 (A proprietary sulfonylurea chemistry compound that enhances kidney ENaC); and

3) 6363969 (A proprietary thio-indole chemistry compound that enhances kidney ENaC).

The results of these experiments are contained in Table 1:

| | | | α1 Splice | |
|---|---|---|---|---|
| Compound | α1βγ | α2βγ | A βγ | Uninject. |
| 3912721 | 93 +/− 27% | 99 +/− 32% | Inactive | Inactive |
| (50 uM) | | | | |
| Pictet Spengler | | | | |
| 8246776 | 299 +/− 65% | 291 +/− 47% | Inactive | Inactive |
| (50 uM) | | | | |
| Sulfonylurea | | | | |
| 6363969 | 1294 +/− 238% | 1033 +/− 153% | Inactive | Inactive |
| (10 uM) | | | | |
| Thio-indole | | | | |

The results summarized in the Table show percent ENaC enhancement values against the three tested EnaC subunit combinations. The results indicate that α1βγ and α2βγ ENaC channels exhibit similar enhancer activation profiles. By contrast, the α1Aβγ splice variant ENaC channels do not exhibit detectable enhancer stimulation and behave similarly to the uninjected oocytes.

### EXAMPLE 4

### Dose-Response Experiments

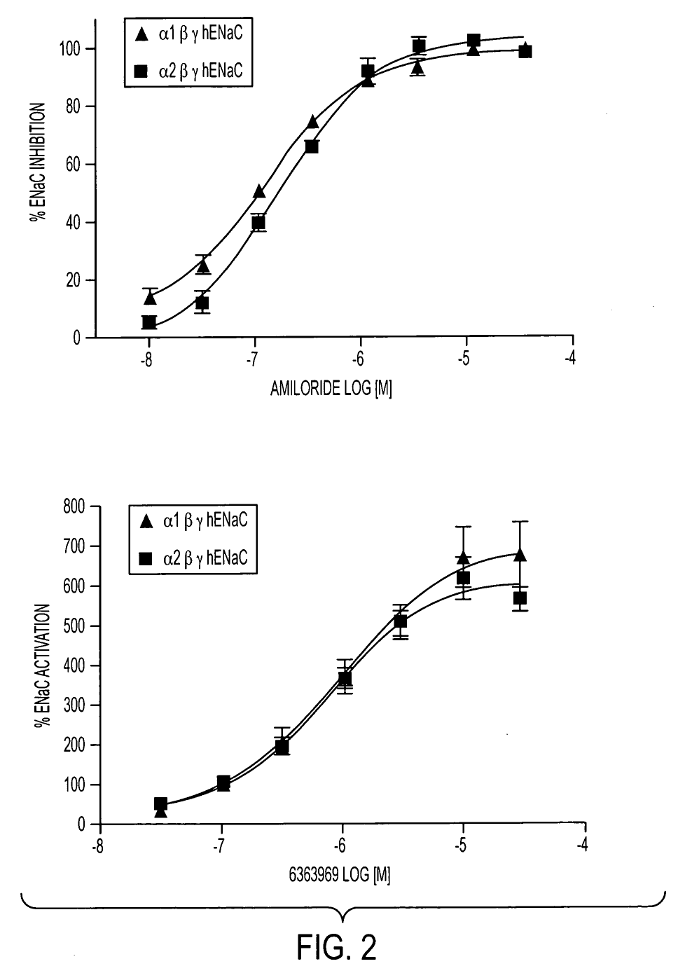

Dose-response experiments were conducted with the blocker amiloride and the proprietary enhancer compound 6363969 on α1βγ and α2βγ ENaC channels. The results (contained in FIG. 2) revealed that the potencies of these compounds were substantially the same for both functional heteromeric channel isoforms. (α1βγ and α2βγ ENaC channels). Particularly, the IC50 for amiloride was 113+/−5 nM for α1βγ ENaC and 153+/−15 nM for α2βγ ENaC. The EC50 for 6363969 was 1.1+/−0.2 μM for α1βγ ENaC and 1.2+/−0.5 μM for α2βγ ENaC.

### CONCLUSIONS

These experiments demonstrate that an ENaC channel expressed in oocytes comprised of α1βγ ENaC subunits functionally similar to α2βγ ENaC with respect to each of basal sodium currents, amiloride inhibition and enhancer stimulation. By contrast, α1βγ ENaC did not generate functional sodium channels in the oocytes systems.

A possible limitation of the experiments conducted to date is the fact that the specific cell(s) from which the splice variants were derived is unknown (because taste tissue used to clone the splice variants was heterogenous and contained non-taste cells). Another possible limitation is that the ENaC channels expressed herein only comprised one splice variant ENaC subunit according to the invention.

With respect to the cell source, in our earlier application and herein the CV papillae preparation used to clone splice variants contained both lingual epithelium (˜90% of material) as well as taste buds (˜10% of material). Accordingly, ENaC splice variants could be derived from a non-taste bud cell source.

Since ENaC is expressed in both lingual and taste tissue in rodents, and since taste tissue comprised a minor fraction of samples used to clone EnaC splice variants, it was anticipated that splice variants derived from taste bud cells would constitute a minor pool of the analyzed clones. In fact, it was observed that most variants (See Example 3) were found in 1-2 of ˜3, clones analyzed.

To conclusively determine what cell type ENaC splice variants are expressed in, in situ hybridization and/or antibody labeling experiments are conducted to examine ENaC expression of the mRNA and protein levels respectively. Alternatively, ENaC splice variants can be isolated from a pure preparation of human taste buds (i.e., containing no lingual epithelial cells), and compared against the splice variants of the present invention.

It is anticipated that the splice variants disclosed herein which are believed to include splice variants expressed in human taste tissue will generate amioride-insensitive channels that mimic or correspond to the primary receptor for salt taste expressed on the human tongue.

These EnaC channels will be exquisitely suitable in the assays described below for identifying modulators of human taste tissue ENaC and salty taste in humans and other mammals.

Assays for Proteins that Modulate ENaC Activity

High throughput functional genomics assays can be used to identify modulators of ENaC which block, inhibit, modulate or enhance salty taste. Such assays can, e.g., monitor changes in cell surface marker expression, changes in intracellular ions, or changes in membrane currents using either cell lines or primary cells. Typically, the cells are contacted with a cDNA or a random peptide library (encoded by nucleic acids). The cDNA library can comprise sense, antisense, full length, and truncated cDNAs. The peptide library is encoded by nucleic acids. The effect of the cDNA or peptide library on the phenotype of the cells is then monitored, using an assay as described above. The effect of the cDNA or peptide can be validated and distinguished from somatic mutations, using, e.g., regulatable expression of the nucleic acid such as expression from a tetracycline promoter. cDNAs and nucleic acids encoding peptides can be rescued using techniques known to those of skill in the art, e.g., using a sequence tag.

Proteins interacting with the peptide or with the protein encoded by the cDNA (e.g., SEQ ID NO: 1, 3, 5, 7, 9, 11, 13, 15, 17, or 19) can be isolated using a yeast two-hybrid system, mammalian two hybrid system, or phage display screen, etc. Targets so identified can be further used as bait in these assays to identify additional components that may interact with the ENaC channel which members are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Suitable cell lines that express ENaC proteins include kidney epithelial cells, lung epithelial cells, taste epithelial cells and other mammalian epithelial cells, preferably human.

Isolation of Nucleic Acids Encoding ENaC Proteins

This invention relies on routine techniques in the field of recombinant genetics. Basic texts disclosing the general methods of use in this invention include Sambrook and Russell, Molecular Cloning, A Laboratory Manual (3^{rd} ed. 2001); Kriegler, Gene Transfer and Expression: A Laboratory Manual (1990); and Current Protocols in Molecular Biology (Ausubel et al., eds., 1994)).

Nucleic acids that encode ENaC subunits, polymorphic variants, orthologs, and alleles that are substantially identical to an amino acid sequence encoded by SEQ ID NO: 2, 4, 6, 8, 10, 12, 14, 16, 18, or 20, as well as other ENaC family members, can be isolated using ENaC nucleic acid probes and oligonucleotides under stringent hybridization conditions, by screening libraries. Alternatively, expression libraries can be used to clone ENaC subunit protein, polymorphic variants, orthologs, and alleles by detecting expressed homologous immunologically with antisera or purified antibodies made against human ENaC or portions thereof.

To make a cDNA library, one should choose a source that is rich in ENaC RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating ENaC subunit nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of human ENaC directly from mRNA, from cDNA, from genomic libraries or cDNA libraries. Degenerate oligonucleotides can be designed to amplify ENaC homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of ENaC encoding mRNA in physiological samples, for nucleic acid sequencing, or for other purposes. Genes amplified by the PCR reaction can be purified from agarose gels and cloned into an appropriate vector.

Gene expression of ENaC subunits can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding ENaC subunit proteins can be used with high-density oligonucleotide array technology (e.g., GeneChip™) to identify ENaC protein, orthologs, alleles, conservatively modified variants, and polymorphic variants in this invention. In the case where the homologs being identified are linked to modulation of T cell activation and migration, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

The genes encoding ENaC subunits preferably human ENaC subunits are typically cloned into intermediate vectors before transformation into prokaryotic or eukaryotic cells for replication and/or expression. These intermediate vectors are typically prokaryotic vectors, e.g., plasmids, or shuttle vectors.

Expression in Prokaryotes and Eukaryotes

To obtain high level expression of a cloned gene, such as those cDNAs encoding hENaC subunit, one typically subclones the hENaC subunit nucleic acid sequence into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al., and Ausubel et al, supra. Bacterial expression systems for expressing the ENaC subunit protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In a preferred embodiment retroviral expression systems are used in the invention. In another embodiment transient expression systems are utilized using plasmid-based vectors that are commercially available such as pcDNA 3 and derivatives thereof.

Selection of the promoter used to direct expression of a heterologous nucleic acid depend on the particular application. The promoter is preferably positioned about the same distance from the heterologous transcription start site, as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the ENaC subunit encoding nucleic acid in host cells. A typical expression cassette thus contains at least one promoter operably linked to a nucleic acid sequence encoding a ENaC subunit(s) and signals required for efficient polyadenylation of the transcript, ribosome binding sites, and translation termination. Additional elements of the cassette may include enhancers and, if genomic DNA is used as the structural gene, introns with functional splice donor and acceptor site.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, .quadrature.-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺ pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a ENaC encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of ENaC protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, lipids optimized for DNA transfection, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one ENaC subunit gene into a host cell, preferably mammalian capable of expressing functional ENaC.

After the expression vector is introduced into the cells, the transfected cells are cultured under conditions favoring expression of ENaC subunit(s). In one embodiment, the cells are transiently transfected with all three hENaC genes using lipid-based transfection and cultured for 24-48 hours prior to performing the screen for ENaC modulators.

Assays for Modulators of ENaC Protein

A. Assays

Modulation of an ENaC protein can be assessed using a variety of assays; preferably cell-based models as described above. Such assays can be used to test for inhibitors and activators of ENaC, which modulate, block, enhance or inhibit salty taste perception.

Preferably, the ENaC will be comprised of three subunits, alpha (or delta), beta and gamma and preferably the human ENaC subunit encoded by the encoded by SEQ ID NO: 1, 3, 5, 7, 9, 11, 13, 15, 17, or 19, or a human ortholog a conservatively modified variant thereof. Alternatively, the ENaC of the assay will be derived from a non-human epithelial cell. Generally, the amino acid sequence identity of each respective subunit will be at least 80%, preferably at least 85%, or 90%, most preferably at least 95%, e.g., 96%, 97%, 98% or 99% to the polypeptide contained in SEQ ID NO: 2, 4, 6, 8, 10, 12, 14, 16, 18, or 20.

Measurement of the effect of a candidate comprised or an ENaC protein or cell expressing ENaC protein, either recombinant or naturally occurring, can be performed using a variety of assays, as described herein. Preferably to identify molecules capable of modulating ENaC, assays are performed to detect the effect of various candidate modulators on ENaC activity in a mammalian cell that expresses a functional ENaC.

The channel activity of ENaC proteins can be assayed using a variety of assays to measure changes in ion fluxes including patch clamp techniques, measurement of whole cell currents, radiolabeled ion flux assays, and fluorescence assays using voltage-sensitive dyes (see, e.g., Vestergarrd-Bogind et al., J. Membrane Biol. 88:67-75 (1988); Daniel et al., J. Pharmacol. Meth. 25:185-193 (1991); Hoevinsky et al., J. Membrane Biol. 137:59-70 (1994)) and ion-sensitive dyes. For example, nucleic acids encoding one or more subunits of an ENaC protein or homologue thereof can be injected into Xenopus oocytes. Channel activity can then be assessed by measuring changes in membrane polarization, i.e., changes in membrane potential. One means to obtain electrophysiological measurements is by measuring currents using patch clamp techniques, e.g., the “cell-attached” mode, the “inside-out” mode, and the “whole cell” mode (see, e.g., Ackerman et al., New Engl. J. Med. 336:1575-1595, 1997). Whole cell currents can be determined using standard methodology such as that described by Hamil et al., PFlugers. Archiv. 391:185 (1981).

Channel activity is also conveniently assessed by measuring changes in intracellular ion levels for example using ion sensitive dyes.

The activity of ENaC polypeptides can be also assessed using a variety of other assays to determine functional, chemical, and physical effects, e.g., measuring the binding of ENaC polypetides to other molecules, including peptides, small organic molecules, and lipids; measuring ENaC protein and/or RNA levels, or measuring other aspects of ENaC polypeptides, e.g., transcription levels, or physiological changes that affects ENaC activity. When the functional consequences are determined using intact cells or animals, one can also measure a variety of effects such as changes in cell growth or pH changes or changes in intracellular second messengers such as IP3, cGMP, or cAMP, or components or regulators of the phospholipase C signaling pathway. Such assays can be used to test for both activators and inhibitors. Modulators thus identified are useful for, e.g., as flavorants in foods, beverages and medicines.

Cell-Based Assays

In another embodiment, at least one ENaC subunit protein is expressed in a cell, and functional, e.g., physical and chemical or phenotypic, changes are assayed to identify ENaC modulators. Cells expressing ENaC proteins can also be used in binding assays. Any suitable functional effect can be measured, as described herein. For example, changes in membrane potential, changes in intracellular ion levels, and ligand binding are all suitable assays to identify potential modulators using a cell based system. Suitable cells for such cell-based assays include both primary cells, e.g., taste epithelial cells that expresses an ENaC protein and cultured cell lines such as HEK293T cells that express an ENaC. The ENaC protein can be naturally occurring or recombinant. Also, as described above, fragments of ENaC proteins or chimeras with ion channel activity can be used in cell based assays.

In another embodiment, cellular ENaC polypeptide levels are determined by measuring the level of protein or mRNA. The level of ENaC protein or proteins related to ENaC ion channel activation are measured using immunoassays such as western blotting, ELISA and the like with an antibody that selectively binds to the ENaC polypeptide or a fragment thereof. For measurement of mRNA, amplification, e.g., using PCR, LCR, or hybridization assays, e.g., Northern hybridization, RNase protection, dot blotting, is preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, ENaC expression can be measured using a reporter gene system. Such a system can be devised using an ENaC protein promoter operably linked to a reporter gene such as chloramphenicol acetyltransferase, firefly luciferase, bacterial luciferase, .beta.-galactosidase and alkaline phosphatase. Furthermore, the protein of interest can be used as an indirect reporter via attachment to a second reporter such as red or green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

In another embodiment, a functional effect related to signal transduction can be measured. An activated or inhibited ENaC will alter the properties of target enzymes, second messengers, channels, and other effector proteins. Assays for ENaC activity include cells that are loaded with ion or voltage sensitive dyes to report channel activity, e.g., by observing membrane depolarization or sodium influx. Assays for determining activity of such receptors can also use known antagonists for ENaC, such as amiloride or phenamil, as controls to assess activity of tested compounds. In assays for identifying modulatory compounds (e.g., agonists, antagonists), changes in the level of ions in the cytoplasm or membrane potential will be monitored using an ion sensitive or membrane potential fluorescent indicator, respectively. Among the ion-sensitive indicators and voltage probes that may be employed are those available from Molecular Probes (See 2002 Catalog: and specific compounds disclosed infra).

Animal Models

Animal models that express hENaC also find use in screening for modulators of salty taste. Similarly, transgenic animal technology including gene knockout technology, for example as a result of homologous recombination with an appropriate gene targeting vector, or gene overexpression, will result in the absence or increased expression of the ENaC protein. The same technology can also be applied to make knockout cells. When desired, tissue-specific expression or knockout of the ENaC protein may be necessary. Transgenic animals generated by such methods find use as animal models of responses to salty taste stimuli.

Knockout cells and transgenic mice can be made by insertion of a marker gene or other heterologous gene into an endogenous ENaC gene site in the mouse genome via homologous recombination. Such mice can also be made by substituting an endogenous ENaC with a mutated version of the ENaC gene, or by mutating an endogenous gene.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

B. Modulators

The compounds tested as modulators of ENaC protein can be any small organic molecule, or a biological entity, such as a protein, e.g., an antibody or peptide, a sugar, a nucleic acid, e.g., an antisense oligonucleotide or a ribozyme, or a lipid. Alternatively, modulators can be genetically altered versions of an ENaC protein. Typically, test compounds will be small organic molecules, peptides, lipids, and lipid analogs. Preferably, the tested compounds are safe for human consumption.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including ChemDiv (San Diego, Calif.), Sigma-Aldrich (St. Louis, Mo.), Fluka Chemika-Biochemica-Analytika (Buchs Switzerland) and the like.

In the preferred embodiment, high throughput screening methods involve providing a small organic molecule or peptide library containing a large number of potential ENaC modulators (potential activator or inhibitor compounds). Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Foods and Beverage Compositions Containing Compound Identified Using Disclosed Assays

The compounds identified using disclosed assays, in particular the fluorescence cell-based assay disclosed in the example, are potentially useful as ingredients or flavorants in ingestible compositions, i.e., foods and beverages as wells as orally administered medicinals. Compounds that modulate or enhance salty taste perception can be used alone or in combination as flavorants in foods or beverages. In the preferred application, the modulator will be incorporated into a food or beverage with a reduced level of sodium and the salty taste of the resulting product will be similar to that of the high sodium product. Examples of such foods and beverages include snack foods such as pretzels, potato chips, crackers, soups, dips, soft drinks, packaged meat products, among others.

Alternatively, compounds that block or inhibit salty taste perception can be used as ingredients or flavorants in foods that naturally contain high salt concentrates in order to block or camouflage the salty taste thereof.

The amount of such compound(s) will be an amount that yields the desired degree of salty taste perception. Of course compounds used in such applications will be determined to be safe for human consumption.

Preferred Embodiment

As disclosed supra preferably, the invention will comprise contacting a test cell expressing a functional ENaC with at least one putative modulator compound in the presence of a membrane potential dye, and monitoring the activity of the ENaC expressed by the test cell to determine the extent of ENaC modulation. The method can further comprise evaluating the putative modulator compound for in vivo effects on salty taste perception (e.g., performing tasting experiments to determine the in vivo effect on salty taste perception). In the preferred embodiment, cDNAs encoding splice variants of ENaC subunits are cloned from human taste cell cDNA. As mentioned above, native ENaC is a multimeric protein consisting of three subunits (alpha or delta, beta, and gamma). ENaC functions as a constitutively active Na⁺ selective cation channel, is found in taste buds as well as other tissues, and is a candidate human salt receptor underlying the physiological perception of salt taste.

In a preferred embodiment, such a method is carried out in a high throughput assay format using multi-well plates and a fluorescence intensity plate reader (e.g., Aurora Biosciences VIPR instrument or Molecular Device's FLIPR instrument). The test cells may be seeded, dye-loaded, contacted with the test compounds, and monitored in the same multi-well plate. Such an assay format can reliably detect both activation or inhibition of ENaC function, providing a robust screen for compounds that could either enhance or block channel activity. The assay described above has been optimized to identify ENaC enhancers. The assay described herein thus has advantages over existing assays, such as those described above, in that a human ENaC is utilized, mammalian cells are employed and the assay can be run in standard multi-well (e.g., 96, 384, or 1536 well) plates in high-throughput mode.

In one aspect of the invention, mammalian cells will be produced that functionally express at least the alpha (or delta) subunit of ENaC. In preferred embodiments, all three subunits of hENaC α or .Δ, β., and γ) are expressed either transiently or stably. The ENaC subunit(s) employed can be naturally occurring forms, variants containing SNPs, alternatively spliced forms, combinations of forms or any functional variants known in the art (see e.g., accession numbers P37088, P51168, P51170, and P51172). Preferably, the ENaC will be comprised of the human alpha, beta and gamma ENaC subunits and will comprise at least one splice variant expressed in human taste bud cells corresponding to the polypeptides encoded by the nucleic acid sequence in SEQ ID NO. 1, 3, 5, 7, 9, 11, 13, 15, 17, or 19, human beta, gamma and delta ENaC subunits having protein sequences contained in SEQ ID NO. 2, 4, 6, 8, 10, 12, 14, 16, 18, or 20. The mammalian cells used for expression can be any type known in the art such as COS, CHO, BHK, MDCK, HEK293, or HEK293T (human embryonic kidney cells expressing the large T-cell antigen). Preferably, the cell is HEK293T. The cells can be transfected using standard methods known in the art, such as but not limited to Ca²⁺ phosphate or lipid-based systems, or methods previously mentioned.

In a preferred embodiment of the invention, transfected cells are seeded into multi-well culture plates. Functional expression is then allowed to proceed for a time sufficient to reach at least about 70% confluence, more preferably to at least about 80% confluence or to form a cell layer dense enough to withstand possible fluid perturbations caused by compound addition. Generally, an incubation time of at least 24 hours will be sufficient, but can be longer as well. The cells are then washed to remove growth media and incubated with a membrane-potential dye for a time sufficient to allow the dye to equilibrate across the plasma membranes of the seeded cells. One of skill in the art will recognize that the dye loading conditions are dependent on factors such as cell type, dye type, incubation parameters, etc. In one embodiment, the dye may be used at about 2 μM to about 5 μM of the final concentration. Further, the optimal dye loading time may range from about 30 to about 60 minutes at 37° C. for most cells. In the preferred embodiment, the membrane potential dyes are from Molecular Devices (cat# R8034). In other embodiments, suitable dyes could include single wavelength-based dyes such as DiBAC, DiSBAC (Molecular Devices), and Di-4-ANEPPS (Biotium), or dual wavelength FRET-based dyes such as DiSBAC2, DiSBAC3, and CC-2-DMPE (Aurora Biosciences). [Chemical Names—Di-4-ANEPPS (Pyridinium, 4-(2-(6-(dibutylamino)-2-naphthalenyl)ethenyl)-1-(3-sulfopro-pyl)-, hydroxide, inner salt), DiSBAC4(2) (bis-(1,2-dibarbituric acid)-trimethine oxanol), DiSBAC4(3) (bis-(1,3-dibarbituric acid)-trimethine oxanol), CC-2-DMPE (Pacific Blue™. 1,2-ditetradecanoyl-sn-glycero-3-phosphoethanolamine, triethylammonium salt) and SBFI-AM (1,3-Benzenedicarboxylic acid, 4,4′-[1,4,10-trioxa-7,13-diazacyclopentadecane-7,13-diylbis(5-methoxy-6,12-benzofurandiyl)]bis-,tetrakis [(acetyloxy)methyl]ester;].

In one embodiment, the dye-loaded cells are then contacted with test compounds (or controls), and the cell cultures are monitored using standard fluorescence analysis instrumentation such as or VIPR or FLIPR™. The addition of NaCl or other test compounds which pharmacologically act on ENaC elicit a change in membrane potential which is then detected as a change in the resting fluorescence in a standard fluorescence intensity plate reader (e.g., FLIPR) or voltage intensity plate reader (e.g. VIPR). As such, the method of the present invention can be used to identify taste modulating compounds by monitoring the activity of ENaC in the test cells through fluorescence. For instance, a decrease in fluorescence may indicate a taste (salty) blocker, while an increase in fluorescence may indicate a taste (salty) enhancer.

Listing of Relevant Nucleic Acid Sequences and Polypeptide ENaC Sequences According to the Invention

Set forth below are nucleic acid sequences and amino acid sequences corresponding to human ENaC alpha, beta and gamma subunits expressed in human kidney and human ENaC alpha, beta and gamma subunit splice variants according to the invention which were cloned from human taste tissue.

DNA Sequences

DNA and Polypeptide ENaC Sequences DNA Sequences

Reference kidney α1 nucleotide sequence (SEQ ID NO:1):

atggaggggaacaagctggaggagcaggactctagccctccacagtccac
tccagggctcatgaaggggaacaagcgtgaggagcaggggctgggccccg
aacctgcggcgccccagcagcccacggcggaggaggaggccctgatcgag
ttccaccgctcctaccgagagctcttcgagttcttctgcaacaacaccac
catccacggcgccatccgcctggtgtgctcccagcacaaccgcatgaaga
cggccttctgggcagtgctgtggctctgcacctttggcatgatgtactgg
caattcggcctgcttttcggagagtacttcagctaccccgtcagcctcaa
catcaacctcaactcggacaagctcgtcttccccgcagtgaccatctgca
ccctcaatccctacaggtacccggaaattaaagaggagctggaggagctg
gaccgcatcacagagcagacgctctttgacctgtacaaatacagctcctt
caccactctcgtggccggctcccgcagccgtcgcgacctgcgggggactc
tgccgcaccccttgcagcgcctgagggtcccgcccccgcctcacggggcc
cgtcgagcccgtagcgtggcctccagcttgcgggacaacaacccccaggt
ggactggaaggactggaagatcggcttccagctgtgcaaccagaacaaat
cggactgcttctaccagacatactcatcaggggtggatgcggtgagggag
tggtaccgcttccactacatcaacatcctgtcgaggctgccagagactct
gccatccctggaggaggacacgctgggcaacttcatcttcgcctgccgct
tcaaccaggtctcctgcaaccaggcgaattactctcacttccaccacccg
atgtatggaaactgctatactttcaatgacaagaacaactccaacctctg
gatgtcttccatgcctggaatcaacaacggtctgtccctgatgctgcgcg
cagagcagaatgacttcattcccctgctgtccacagtgactggggcccgg
gtaatggtgcacgggcaggatgaacctgcctttatggatgatggtggctt
taacttgcggcctggcgtggagacctccatcagcatgaggaaggaaaccc
tggacagacttgggggcgattatggcgactgcaccaagaatggcagtgat
gttcctgttgagaacctttacccttcaaagtacacacagcaggtgtgtat
tcactcctgcttccaggagagcatgatcaaggagtgtggctgtgcctaca
tcttctatccgcggccccagaacgtggagtactgtgactacagaaagcac
agttcctgggggtactgctactataagctccaggttgacttctcctcaga
ccacctgggctgtttcaccaagtgccggaagccatgcagcgtgaccagct
accagctctctgctggttactcacgatggccctcggtgacatcccaggaa
tgggtcttccagatgctatcgcgacagaacaattacaccgtcaacaacaa
gagaaatggagtggccaaagtcaacatcttcttcaaggagctgaactaca
aaaccaattctgagtctccctctgtcacgatggtcaccctcctgtccaac
ctgggcagccagtggagcctgtggttcggctcctcggtgttgtctgtggt
ggagatggctgagctcgtctttgacctgctggtcatcatgttcctcatgc
tgctccgaaggttccgaagccgatactggtctccaggccgagggggcagg
ggtgctcaggaggtagcctccaccctggcatcctcccctccttcccactt
ctgcccccaccccatgtctctgtccttgtcccagccaggccctgctccct
ctccagccttgacagcccctccccctgcctatgccaccctgggcccccgc
ccatctccagggggctctgcaggggccagttcctccacctgtcctctggg
ggggccctgagagggaaggagaggtttctcacaccaaggcagatgctcct
ctggtgggagggtgctggccctggcaagattgaaggatgtgcaggaattc

Predicted kidney α1 protein sequence (SEQ ID NO:2):

MEGNKLEEQDSSPPQSTPGLMKGNKREEQGLGPEPAAPQQPTAEEEALIE
FHRSYRELFEFFCNNTTIHGAIRLVCSQHNRMKTAFWAVLWLCTFGMMYW
QFGLLFGEYFSYPVSLNINLNSDKLVFPAVTICTLNPYRYPEIKEELEEL
DRITEQTLFDLYKYSSFTTLVAGSRSRRDLRGTLPHPLQRLRVPPPPHGA
RRARSVASSLRDNNPQVDWKDWKIGFQLCNQNKSDCFYQTYSSGVDAVRE
WYRFHYINILSRLPETLPSLEEDTLGNFIFACRFNQVSCNQANYSHFHHP
MYGNCYTFNDKNNSNLWMSSMPGINNGLSLMLRAEQNDFIPLLSTVTGAR
VMVHGQDEPAFMDDGGFNLRPGVETSISMRKETLDRLGGDYGDCTKNGSD
VPVENLYPSKYTQQVCIHSCFQESMIKECGCAYIFYPRPQNVEYCDYRKH
SSWGYCYYKLQVDFSSDHLGCFTKCRKPCSVTSYQLSAGYSRWPSVTSQE
WVFQMLSRQNNYTVNNKRNGVAKVNIFFKELNYKTNSESPSVTMVTLLSN
LGSQWSLWFGSSVLSVVEMAELVFDLLVIMFLMLLRRFRSRYWSPGRGGR
GAQEVASTLASSPPSHFCPHPMSLSLSQPGPAPSPALTAPPPAYATLGPR
PSPGGSAGASSSTCPLGGP

α1A splice variant nucleotide sequence (SEQ ID NO:3):

atggaggggaacaagctggaggagcaggactctagccctccacagtccac
tccagggctcatgaaggggaacaagcgtgaggagcaggggctgggccccg
aacctgcggcgccccagcagcccacggcggaggaggaggccctgatcgag
ttccaccgctcctaccgagagctcttcgagttcttctgcaacaacaccac
catccacggcgccatccgcctggtgtgctcccagcacaaccgcatgaaga
cggccttctgggcagtgctgtggctctgcacctttggcatgatgtactgg
caattcggcctgcttttcggagagtacttcagctaccccgtcagcctcaa
catcaacctcaactcggacaagctcgtcttccccgcagtgaccatctgca
ccctcaatccctacaggtacccggaaattaaagaggagctggaggagctg
gaccgcatcacagagcagacgctctttgacctgtacaaatacagctcctt
caccactctcgtggccggctcccgcagccgtcgcgacctgcgggggactc
tgccgcaccccttgcagcgcctgagggtcccgcccccgcctcacggggcc
cgtcgagcccgtagcgtggcctccagcttgcgggacaacaacccccaggt
ggactggaaggactggaagatcggcttccagctgtgcaaccagaacaaat
cggactgcttctaccagacatactcatcaggggtggatgcggtgagggag
tggtaccgcttccactacatcaacatcctgtcgaggctgccagagactct
gccatccctggaggaggacacgctgggcaacttcatcttcgcctgccgct
tcaaccaggtctcctgcaaccaggcgaattactctcacttccaccacccg
atgtatggaaactgctatactttcaatgacaagaacaactccaacctctg
gatgtcttccatgcctggaatcaacaacgtgactggggcccgggtaatgg
tgcacgggcaggatgaacctgcctttatggatgatggtggctttaacttg
cggcctggcgtggagacctccatcagcatgaggaaggaaaccctggacag
acttgggggcgattatggcgactgcaccaagaatggcagtgatgttcctg
ttgagaacctttacccttcaaagtacacacagcaggtgtgtattcactcc
tgcttccaggagagcatgatcaaggagtgtggctgtgcctacatcttcta
tccgcggccccagaacgtggagtactgtgactacagaaagcacagttcct
gggggtactgctactataagctccaggttgacttctcctcagaccacctg
ggctgtttcaccaagtgccggaagccatgcagcgtgaccagctaccagct
ctctgctggttactcacgatggccctcggtgacatcccaggaatgggtct
tccagatgctatcgcgacagaacaattacaccgtcaacaacaagagaaat
ggagtggccaaagtcaacatcttcttcaaggagctgaactacaaaaccaa
ttctgagtctccctctgtcacgatggtcaccctcctgtccaacctgggca
gccagtggagcctgtggttcggctcctcggtgttgtctgtggtggagatg
gctgagctcgtctttgacctgctggtcatcatgttcctcatgctgctccg
aaggttccgaagccgatactggtctccaggccgagggggcaggggtgctc
aggaggtagcctccaccctggcatcctcccctccttcccacttctgcccc
caccccatgtctctgtccttgtcccagccaggccctgctccctctccagc
cttgacagcccctccccctgcctatgccaccctgggcccccgcccatctc
cagggggctctgcaggggccagttcctccacctgtcctctgggggggccc
tga

α1A splice variant predicted protein sequence (SEQ ID NO:4):

MEGNKLEEQDSSPPQSTPGLMKGNKREEQGLGPEPAAPQQPTAEEEALIE
FHRSYRELFEFFCNNTTIHGAIRLVCSQHNRMKTAFWAVLWLCTFGMMYW
QFGLLFGEYFSYPVSLNINLNSDKLVFPAVTICTLNPYRYPEIKEELEEL
DRITEQTLFDLYKYSSFTTLVAGSRSRRDLRGTLPHPLQRLRVPPPPHGA
RRARSVASSLRDNNPQVDWKDWKIGFQLCNQNKSDCFYQTYSSGVDAVRE
WYRFHYINILSRLPETLPSLEEDTLGNFIFACRFNQVSCNQANYSHFHHP
MYGNCYTFNDKNNSNLWMSSMPGINNVTGARVMVHGQDEPAFMDDGGFNL
RPGVETSISMRKETLDRLGGDYGDCTKNGSDVPVENLYPSKYTQQVCIHS
CFQESMIKECGCAYIFYPRPQNVEYCDYRKHSSWGYCYYKLQVDFSSDHL
GCFTKCRKPCSVTSYQLSAGYSRWPSVTSQEWVFQMLSRQNNYTVNNKRN
GVAKVNIFFKELNYKTNSESPSVTMVTLLSNLGSQWSLWFGSSVLSVVEM
AELVFDLLVIMFLMLLRRFRSRYWSPGRGGRGAQEVASTLASSPPSHFCP
HPMSLSLSQPGPAPSPALTAPPPAYATLGPRPSPGGSAGASSSTCPLGGP

α2 reference nucleotide sequence (SEQ ID NO:5):

atgggcatggccaggggcagcctcactcgggttccaggggtgatgggaga
gggcactcagggcccagagctcagccttgaccctgacccttgctctcccc
aatccactccggggctcatgaaggggaacaagctggaggagcaggaccct
agacctctgcagcccataccaggtctcatggaggggaacaagctggagga
gcaggactctagccctccacagtccactccagggctcatgaaggggaaca
agcgtgaggagcaggggctgggccccgaacctgcggcgccccagcagccc
acggcggaggaggaggccctgatcgagttccaccgctcctaccgagagct
cttcgagttcttctgcaacaacaccaccatccacggcgccatccgcctgg
tgtgctcccagcacaaccgcatgaagacggccttctgggcagtgctgtgg
ctctgcacctttggcatgatgtactggcaattcggcctgcttttcggaga
gtacttcagctaccccgtcagcctcaacatcaacctcaactcggacaagc
tcgtcttccccgcagtgaccatctgcaccctcaatccctacaggtacccg
gaaattaaagaggagctggaggagctggaccgcatcacagagcagacgct
ctttgacctgtacaaatacagctccttcaccactctcgtggccggctccc
gcagccgtcgcgacctgcgggggactctgccgcaccccttgcagcgcctg
agggtcccgcccccgcctcacggggcccgtcgagcccgtagcgtggcctc
cagcttgcgggacaacaacccccaggtggactggaaggactggaagatcg
gcttccagctgtgcaaccagaacaaatcggactgcttctaccagacatac
tcatcaggggtggatgcggtgagggagtggtaccgcttccactacatcaa
catcctgtcgaggctgccagagactctgccatccctggaggaggacacgc
tgggcaacttcatcttcgcctgccgcttcaaccaggtctcctgcaaccag
gcgaattactctcacttccaccacccgatgtatggaaactgctatacttt
caatgacaagaacaactccaacctctggatgtcttccatgcctggaatca
acaacggtctgtccctgatgctgcgcgcagagcagaatgacttcattccc
ctgctgtccacagtgactggggcccgggtaatggtgcacgggcaggatga
acctgcctttatggatgatggtggctttaacttgcggcctggcgtggaga
cctccatcagcatgaggaaggaaaccctggacagacttgggggcgattat
ggcgactgcaccaagaatggcagtgatgttcctgttgagaacctttaccc
ttcaaagtacacacagcaggtgtgtattcactcctgcttccaggagagca
tgatcaaggagtgtggctgtgcctacatcttctatccgcggccccagaac
gtggagtactgtgactacagaaagcacagttcctgggggtactgctacta
taagctccaggttgacttctcctcagaccacctgggctgtttcaccaagt
gccggaagccatgcagcgtgaccagctaccagctctctgctggttactca
cgatggccctcggtgacatcccaggaatgggtcttccagatgctatcgcg
acagaacaattacaccgtcaacaacaagagaaatggagtggccaaagtca
acatcttcttcaaggagctgaactacaaaaccaattctgagtctccctct
gtcacgatggtcaccctcctgtccaacctgggcagccagtggagcctgtg
gttcggctcctcggtgttgtctgtggtggagatggctgagctcgtctttg
acctgctggtcatcatgttcctcatgctgctccgaaggttccgaagccga
tactggtctccaggccgagggggcaggggtgctcaggaggtagcctccac
cctggcatcctcccctccttcccacttctgcccccaccccatgtctctgt
ccttgtcccagccaggccctgctccctctccagccttgacagcccctccc
cctgcctatgccaccctgggcccccgcccatctccagggggctctgcagg
ggccagttcctccacctgtcctctgggggggccctga

α2 reference predicted protein sequence (SEQ ID NO:6):

MGMARGSLTRVPGVMGEGTQGPELSLDPDPCSPQSTPGLMKGNKLEEQDP
RPLQPIPGLMEGNKLEEQDSSPPQSTPGLMKGNKREEQGLGPEPAAPQQP
TAEEEALIEFHRSYRELFEFFCNNTTIHGAIRLVCSQHNRMKTAFWAVLW
LCTFGMMYWQFGLLFGEYFSYPVSLNINLNSDKLVFPAVTICTLNPYRYP
EIKEELEELDRITEQTLFDLYKYSSFTTLVAGSRSRRDLRGTLPHPLQRL
RVPPPPHGARRARSVASSLRDNNPQVDWKDWKIGFQLCNQNKSDCFYQTY
SSGVDAVREWYRFHYINILSRLPETLPSLEEDTLGNFIFACRFNQVSCNQ
ANYSHFHHPMYGNCYTFNDKNNSNLWMSSMPGINNGLSLMLRAEQNDFIP
LLSTVTGARVMVHGQDEPAFMDDGGFNLRPGVETSISMRKETLDRLGGDY
GDCTKNGSDVPVENLYPSKYTQQVCIHSCFQESMIKECGCAYIFYPRPQN
VEYCDYRKHSSWGYCYYKLQVDFSSDHLGCFTKCRKPCSVTSYQLSAGYS
RWPSVTSQEWVFQMLSRQNNYTVNNKRNGVAKVNIFFKELNYKTNSESPS
VTMVTLLSNLGSQWSLWFGSSVLSVVEMAELVFDLLVIMFLMLLRRFRSR
YWSPGRGGRGAQEVASTLASSPPSHFCPHPMSLSLSQPGPAPSPALTAPP
PAYATLGPRPSPGGSAGASSSTCPLGGP

α2A splice variant nucleotide sequence (SEQ ID NO:7):

atgggcatggccaggggcagcctcactcgggttccaggggtgatgggaga
gggcactcagggcccagagctcagccttgaccctgacccttgctctcccc
aatccactccggggctcatgaaggggaacaagctggaggagcaggaccct
agacctctgcagcccataccaggtctcatggaggggaacaagctggagga
gcaggactctagccctccacagtccactccagggctcatgaaggggaaca
agcgtgaggagcaggggctgggccccgaacctgcggcgccccagcagccc
acggcggaggaggaggccctgatcgagttccaccgctcctaccgagagct
cttcgagttcttctgcaacaacaccaccatccacggcgccatccgcctgg
tgtgctcccagcacaaccgcatgaagacggccttctgggcagtgctgtgg
ctctgcacctttggcatgatgtactggcaattcggcctgcttttcggaga
gtacttcagctaccccgtcagcctcaacatcaacctcaactcggacaagc
tcgtcttccccgcagtgaccatctgcaccctcaatccctacaggtacccg
gaaattaaagaggagctggaggagctggaccgcatcacagagcagacgct
ctttgacctgtacaaatacagctccttcaccactctcgtggccggctccc
gcagccgtcgcgacctgcgggggactctgccgcaccccttgcagcgcctg
agggtcccgcccccgcctcacggggcccgtcgagcccgtagcgtggcctc
cagcttgcgggacaacaacccccaggtggactggaaggactggaagatcg
gcttccagctgtgcaaccagaacaaatcggactgcttctaccagacatac
tcatcaggggtggatgcggtgagggagtggtaccgcttccactacatcaa
catcctgtcgaggctgccagagactctgccatccctggaggaggacacgc
tgggcaacttcatcttcgcctgccgcttcaaccaggtctcctgcaaccag
gcgaattactctcacttccaccacccgatgtatggaaactgctatacttt
caatgacaagaacaactccaacctctggatgtcttccatgcctggaatca
acaacgtgactggggcccgggtaatggtgcacgggcaggatgaacctgcc
tttatggatgatggtggctttaacttgcggcctggcgtggagacctccat
cagcatgaggaaggaaaccctggacagacttgggggcgattatggcgact
gcaccaagaatggcagtgatgttcctgttgagaacctttacccttcaaag
tacacacagcaggtgtgtattcactcctgcttccaggagagcatgatcaa
ggagtgtggctgtgcctacatcttctatccgcggccccagaacgtggagt
actgtgactacagaaagcacagttcctgggggtactgctactataagctc
caggttgacttctcctcagaccacctgggctgtttcaccaagtgccggaa
gccatgcagcgtgaccagctaccagctctctgctggttactcacgatggc
cctcggtgacatcccaggaatgggtcttccagatgctatcgcgacagaac
aattacaccgtcaacaacaagagaaatggagtggccaaagtcaacatctt
cttcaaggagctgaactacaaaaccaattctgagtctccctctgtcacga
tggtcaccctcctgtccaacctgggcagccagtggagcctgtggttcggc
tcctcggtgttgtctgtggtggagatggctgagctcgtctttgacctgct
ggtcatcatgttcctcatgctgctccgaaggttccgaagccgatactggt
ctccaggccgagggggcaggggtgctcaggaggtagcctccaccctggca
tcctcccctccttcccacttctgcccccaccccatgtctctgtccttgtc
ccagccaggccctgctccctctccagccttgacagcccctccccctgcct
atgccaccctgggcccccgcccatctccagggggctctgcaggggccagt
tcctccacctgtcctctgggggggccctga

α2A splice variant predicted protein sequence (SEQ ID NO:8):

MGMARGSLTRVPGVMGEGTQGPELSLDPDPCSPQSTPGLMKGNKLEEQDP
RPLQPIPGLMEGNKLEEQDSSPPQSTPGLMKGNKREEQGLGPEPAAPQQP
TAEEEALIEFHRSYRELFEFFCNNTTIHGAIRLVCSQHNRMKTAFWAVLW
LCTFGMMYWQFGLLFGEYFSYPVSLNINLNSDKLVFPAVTICTLNPYRYP
EIKEELEELDRITEQTLFDLYKYSSFTTLVAGSRSRRDLRGTLPHPLQRL
RVPPPPHGARRARSVASSLRDNNPQVDWKDWKIGFQLCNQNKSDCFYQTY
SSGVDAVREWYRFHYINILSRLPETLPSLEEDTLGNFIFACRFNQVSCNQ
ANYSHFHHPMYGNCYTFNDKNNSNLWMSSMPGINNVTGARVMVHGQDEPA
FMDDGGFNLRPGVETSISMRKETLDRLGGDYGDCTKNGSDVPVENLYPSK
YTQQVCIHSCFQESMIKECGCAYIFYPRPQNVEYCDYRKHSSWGYCYYKL
QVDFSSDHLGCFTKCRKPCSVTSYQLSAGYSRWPSVTSQEWVFQMLSRQN
NYTVNNKRNGVAKVNIFFKELNYKTNSESPSVTMVTLLSNLGSQWSLWFG
SSVLSVVEMAELVFDLLVIMFLMLLRRFRSRYWSPGRGGRGAQEVASTLA
SSPPSHFCPHPMSLSLSQPGPAPSPALTAPPPAYATLGPRPSPGGSAGAS
SSTCPLGGP

β ENaC kidney reference nucleotide sequence (SEQ ID NO:9):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcggggtactcgtggacaag
cttcagcgcatgggggagccctacagcccgtgcaccgtgaatggttctga
ggtccccgtccaaaacttctacagtgactacaacacgacctactccatcc
aggcctgtcttcgctcctgcttccaagaccacatgatccgtaactgcaac
tgtggccactacctgtacccactGccccgtggggagaaatactgcaacaa
ccgggacttcccagactgggcccattgctactcagatctacagatgagcg
tggcgcagagagagacctgcattggcatgtgcaaggagtcctgcaatgac
acccagtacaagatgaccatctccatggctgactggccttctgaggcctc
cgaggactggattttccacgtcttgtctcaggagcgggaccaaagcacca
atatcaccctgagcaggaagggaattgtcaagctcaacatctActtccaa
gaatttaactatcgcaccattgaagaatcagcagccaataacatcgtctg
gctgctctcgaatctgggtggccagtttggcttctggatggggggctctg
tgctgtgcctcatcgagtttggggagatcatcatcgactttgtgtggatc
accatcatcaagctggtggccttggccaagagcctacggcagcggcgagc
ccaagccagCtacgctggcccaccgcccaccgtggccgagctggtggagg
cccacaccaactttggcttccagcctgacacggccccccgcagccccaac
actgggccctaccccagtgagcaggccctgcccatcccaggcaccccgcc
ccccaactatgactccctgcgtctgcagccgctggacgtcatcgagtctg
acagtgagggtgatgccatctaa

β ENaC kidney reference predicted protein sequence (SEQ ID NO:10):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICN
ASPFKYSKIKHLLKDLDELMEAVLERILAPELSHANATRNLNFSIWNHT
PLVLIDERNPHHPMVLDLFGDNHNGLTSSSASEKICNAHGCKMAMRLCS
LNRTQCTFRNFTSATQALTEWYILQATNIFAQVPQQELVEMSYPGEQMI
LACLFGAEPCNYRNFTSIFYPHYGNCYIFNWGMTEKALPSANPGTEFGL
KLILDIGQEDYVPFLASTAGVRLMLHEQRSYPFIRDEGIYAMSGTETSI
GVLVDKLQRMGEPYSPCTVNGSEVPVQNFYSDYNTTYSIQACLRSCFQD
HMIRNCNCGHYLYPLPRGEKYCNNRDFPDWAHCYSDLQMSVAQRETCIG
MCKFSCNDTQYKMTISMADWPSEASEDWIFHVLSQERDQSTNITLSRKG
IVKLNIYFQEFNYRTIEESAANNIVWLLSNLGGQFGFWMGGSVLCLIEF
GEIIIDFVWITIIKLVALAKSLRQRRAQASYAGPPPTVAELVEAHTNFG
FQPDTAPRSPNTGPYPSEQALPIPGTPPPNYDSLRLQPLDVIESDSEGD
AI

βA splice variant nucleotide sequence (SEQ ID NO:11):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcggggtactcgtggcctgt
cttcgctcctgcttccaagaccacatgatccgtaactgcaactgtggcca
ctacctgtacccactGccccgtggggagaaatactgcaacaaccgggact
tcccagactgggcccattgctactcagatctacagatgagcgtggcgcag
agagagacctgcattggcatgtgcaaggagtcctgcaatgacacccagta
caagatgaccatctccatggctgactggccttctgaggcctccgaggact
ggattttccacgtcttgtctcaggagcgggaccaaagcaccaatatcacc
ctgagcaggaagggaattgtcaagctcaacatctActtccaagaatttaa
ctatcgcaccattgaagaatcagcagccaataacatcgtctggctgctct
cgaatctgggtggccagtttggcttctggatggggggctctgtgctgtgc
ctcatcgagtttggggagatcatcatcgactttgtgtggatcaccatcat
caagctggtggccttggccaagagcctacggcagcggcgagcccaagcca
gCtacgctggcccaccgcccaccgtggccgagctggtggaggcccacacc
aactttggcttccagcctgacacggccccccgcagccccaacactgggcc
ctaccccagtgagcaggccctgcccatcccaggcaccccgccccccaact
atgactccctgcgtctgcagccgctggacgtcatcgagtctgacagtgag
ggtgatgccatctaa

βB splice variant predicted protein sequence (SEQ ID NO:12):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAQVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGVLVAC
LRSCFQDHMIRNCNCGHYLYPLPRGEKYCNNRDFPDWAHCYSDLQMSVAQ
RETCIGMCKESCNDTQYKMTISMADWPSEASEDWIFHVLSQERDQSTNIT
LSRKGIVKLNIYFQEFNYRTIEESAANNIVWLLSNLGGQFGFWMGGSVLC
LIEFGEIIIDFVWITIIKLVALAKSLRQRRAQASYAGPPPTVAELVEAHT
NFGFQPDTAPRSPNTGPYPSEQALPIPGTPPPNYDSLRLQPLDVIESDSE
GDAI

βB splice variant nucleotide sequence (SEQ ID NO:13):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagaacttcacgtccatcttctaccctcactatggcaactg
ttacatcttcaactggggcatgacagagaaggcacttccttcggccaacc
ctggaactgaattcggcctgaagttgatcctggacataggccaggaagac
tacgtccccttccttgcgtccacggccggggtcaggctgatgcttcacga
gcagaggtcataccccttcatcagagatgagggcatctacGccatgtcgg
ggacagagacgtccatcgggtactcgtggacaagcttcagcgcatggggg
agccctacagcccgtgcaccgtgaatggttctgaggtccccgtccaaaac
ttctacagtgactacaacacgacctactccatccaggcctgtcttcgctc
ctgcttccaagaccacatgatccgtaactgcaactgtggccactacctgt
acccactGccccgtggggagaaatactgcaacaaccgggacttcccagac
tgggcccattgctactcagatctacagatgagcgtggcgcagagagagac
ctgcattggcatgtgcaaggagtcctgcaatgacacccagtacaagatga
ccatctccatggctgactggccttctgaggcctccgaggactggattttc
cacgtcttgtctcaggagcgggaccaaagcaccaatatcaccctgagcag
gaagggaattgtcaagctcaacatctActtccaagaatttaactatcgca
ccattgaagaatcagcagccaataacatcgtctggctgctctcgaatctg
ggtggccagtttggcttctggatggggggctctgtgctgtgcctcatcga
gtttggggagatcatcatcgactttgtgtggatcaccatcatcaagctgg
tggccttggccaagagcctacggcagcggcgagcccaagccagCtacgct
ggcccaccgcccaccgtggccgagctggtggaggcccacaccaactttgg
cttccagcctgacacggccccccgcagccccaacactgggccctacccca
gtgagcaggccctgcccatcccaggcaccccgccccccaactatgactcc
ctgcgtctgcagccgctggacgtcatcgagtctgacagtgagggtgatgc
catctaa

βB splice variant predicted protein sequence (SEQ ID NO:14):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKNFTSIFYPHYGNCYIFNWGMTEKALPSANPGTEFGLKLILDIGQED
YVPFLASTAGVRLMLHEQRSYPFIRDEGIYAMSGTETSIGVLVDKLQRMG
EPYSPCTVNGSEVPVQNFYSDYNTTYSIQACLRSCFQDHMIRNCNCGHYL
YPLPRGEKYCNNRDFPDWAHCYSDLQMSVAQRETCIGMCKESCNDTQYKM
TISMADWPSEASEDWIFHVLSQERDQSTNITLSRKGIVKLNIYFQEFNYR
TIEESAANNIVWLLSNLGGQFGFWMGGSVLCLIEFGEIIIDFVWITIIKL
VALAKSLRQRRAQASYAGPPPTVAELVEAHTNFGFQPDTAPRSPNTGPYP
SEQALPIPGTPPPNYDSLRLQPLDVIESDSEGDAI

β* splice variant nucleotide sequence (SEQ ID NO:15):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcgggGACaagcttcagcgc
atgggggagccctacagcccgtgcaccgtgaatggttctgaggtccccgt
ccaaaacttctacagtgactacaacacgacctactccatccaggcctgtc
ttcgctcctgcttccaagaccacatgatccgtaactgcaactgtggccac
tacctgtacccactGccccgtggggagaaatactgcaacaaccgggactt
cccagactgggcccattgctactcagatctacagatgagcgtggcgcaga
gagagacctgcattggcatgtgcaaggagtcctgcaatgacacccagtac
aagatgaccatctccatggctgactggccttctgaggcctccgaggactg
gattttccacgtcttgtctcaggagcgggaccaaagcaccaatatcaccc
tgagcaggaagggaattgtcaagctcaacatctActtccaagaatttaac
tatcgcaccattgaagaatcagcagccaataacatcgtctggctgctctc
gaatctgggtggccagtttggcttctggatggggggctctgtgctgtgcc
tcatcgagtttggggagatcatcatcgactttgtgtggatcaccatcatc
aagctggtggccttggccaagagcctacggcagcggcgagcccaagccag
Ctacgctggcccaccgcccaccgtggccgagctggtggaggcccacacca
actttggcttccagcctgacacggccccccgcagccccaacactgggccc
taccccagtgagcaggccctgcccatcccaggcaccccgccccccaacta
tgactccctgcgtctgcagccgctggacgtcatcgagtctgacagtgagg
gtgatgccatctaa

β* splice variant predicted protein sequence (SEQ ID NO:16):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAQVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGdKLQR
MGEPYSPCTVNGSEVPVQNFYSDYNTTYSIQACLRSCFQDHMIRNCNCGH
YLYPLPRGEKYCNNRDFPDWAHCYSDLQMSVAQRETCIGMCKESCNDTQY
KMTISMADWPSEASEDWIFHVLSQERDQSTNITLSRKGIVKLNIYFQEFN
YRTIEESAANNIVWLLSNLGGQFGFWMGGSVLCLIEFGEIIIDFVWITII
KLVALAKSLRQRRAQASYAGPPPTVAELVEAHTNFGFQPDTAPRSPNTGP
YPSEQALPIPGTPPPNYDSLRLQPLDVIESDSEGDAI

β** splice variant nucleotide sequence (SEQ ID NO:17):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcggggtactcGacaagctt
cagcgcatgggggagccctacagcccgtgcaccgtgaatggttctgaggt
ccccgtccaaaacttctacagtgactacaacacgacctactccatccagg
cctgtcttcgctcctgcttccaagaccacatgatccgtaactgcaactgt
ggccactacctgtacccactGccccgtggggagaaatactgcaacaaccg
ggacttcccagactgggcccattgctactcagatctacagatgagcgtgg
cgcagagagagacctgcattggcatgtgcaaggagtcctgcaatgacacc
cagtacaagatgaccatctccatggctgactggccttctgaggcctccga
ggactggattttccacgtcttgtctcaggagcgggaccaaagcaccaata
tcaccctgagcaggaagggaattgtcaagctcaacatctActtccaagaa
tttaactatcgcaccattgaagaatcagcagccaataacatcgtctggct
gctctcgaatctgggtggccagtttggcttctggatggggggctctgtgc
tgtgcctcatcgagtttggggagatcatcatcgactttgtgtggatcacc
atcatcaagctggtggccttggccaagagcctacggcagcggcgagccca
agccagCtacgctggcccaccgcccaccgtggccgagctggtggaggccc
acaccaactttggcttccagcctgacacggccccccgcagccccaacact
gggccctaccccagtgagcaggccctgcccatcccaggcaccccgccccc
caactatgactccctgcgtctgcagccgctggacgtcatcgagtctgaca
gtgagggtgatgccatctaa

β** splice variant predicted protein sequence (SEQ ID NO:18):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAQVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGVLdKL
QRMGEPYSPCTVNGSEVPVQNFYSDYNTTYSIQACLRSCFQDHMIRNCNC
GHYLYPLPRGEKYCNNRDFPDWAHCYSDLQMSVAQRETCIGMCKESCNDT
QYKMTISMADWPSEASEDWIFHVLSQERDQSTNITLSRKGIVKLNIYFQE
FNYRTIEESAANNIVWLLSNLGGQFGFWMGGSVLCLIEFGEIIIDFVWIT
IIKLVALAKSLRQRRAQASYAGPPPTVAELVEAHTNFGFQPDTAPRSPNT
GPYPSEQALPIPGTPPPNYDSLRLQPLDVIESDSEGDAI

γ ENaC kidney reference nucleotide sequence (SEQ ID NO:19):

atggcacccggagagaagatcaaagccaaaatcaagaagaatctgcccgt
gacgggccctcaggcgccgaccattaaagagctgatgcggtggtactgcc
tcaacaccaacacccatggctgtcgccgcatcgtggtgtcccgcggccgt
ctgcgccgcctcctctggatcgggttcacactgactgccgtggccctcat
cctctggcagtgcgccctcctcgtcttctccttctatactgtctcagttt
ccatcaaagtccacttccggaagctggattttcctgcagtcaccatctgc
aacatcaacccctacaagtacagcaccgttcgccaccttctagctgactt
ggaacaggagaccagagaggccctgaagtccctgtatggctttccagagt
cccggaagcgccgagaggcggagtcctggaactccgtctcagagggaaag
cagcctagattctcccaccggattccgctgctgatctttgatcaggatga
gaagggcaaggccagggacttcttcacagggAggaagcggaaagtcggcg
gtagcatcattcacaaggcttcaaatgtcatgcacatcgagtccaagcaa
gtggtgggattccaactgtgctcaaatgacacctccgactgtgccaccta
caccttcagctcgggaatcaatgccattcaggagtggtataagctacact
acatgaacatcatggcacaggtgcctctggagaagaaaatcaacatgagc
tattctgctgaggagctgctggtgacctgcttctttgatggagtgtcctg
tgatgccaggaatttcacgcttttCcaccacccgatgcatgggaattgct
atactttcaacaacagagaaaatgagaccattctcagcacctccatgggg
ggcagcgaatatgggctgcaagtcattttgtacataaacgaagaggaata
caacccattcctcgtgtcctccactggagctaaggtgatcatccatcggc
aggatgagtatcccttcgtcgaagatgtgggaacagagattgagacagca
atggtcacctctataggaatgcacctgacagagtccttcaagctgagtga
gccctacagtcagtgcacggaggacgggagtgacgtgccaatcaggaaca
tctacaacgctgcctactcgctccagatctgccttcattcatgcttccag
acaaagatggtggagaaatgtgggtgtgcccagtacagccagcctctacc
tcctgcagccaactactgcaactaccagcagcaccccaactggatgtatt
gttactaccaactgcatcgagcctttgtccaggaagagctgggctgccag
tctgtgtgcaaggaagcctgcagctttaaagagtggacactaaccacaag
cctggcacaatggccatctgtggtttcggagaagtggttgctgcctgttc
tcacttgggaccaaggccggcaagtaaacaaaaagctcaacaagacagac
ttgGccaaactcttgatattctacaaagacctgaaccagagatccatcat
ggagagcccagccaacagtattgagatgcttctgtccaacttcggtggcc
agctgggcctgtggatgagctgctctgttgtctgcgtcatcgagatcatc
gaggtcttcttcattgacttcttctctatcattgcccgccgccagtggca
gaaagccaaggagtggtgggcctggaaacaggctcccccatgtccagaag
ctccccgtagcccacagggccaggacaatccagccctggatatagacgat
gacctacccactttcaactctgctttgcacctgcctccaGccctaggaac
ccaagtgcccggcacaccgccccccaaatacaataccttgcgcttggaga
gggccttttccaaccagctcacagatacccagatgctAgatgagctctga

γ ENaC kidney reference predicted protein sequence (SEQ ID NO:20):

MAPGEKIKAKIKKNLPVTGPQAPTIKELMRWYCLNTNTHGCRRIVVSRGR
LRRLLWIGFTLTAVALILWQCALLVFSFYTVSVSIKVHFRKLDFPAVTIC
NINPYKYSTVRHLLADLEQETREALKSLYGFPESRKRREAESWNSVSEGK
QPRFSHRIPLLIFDQDEKGKARDFFTGRKRKVGGSIIHKASNVMHIESKQ
VVGFQLCSNDTSDCATYTFSSGINAIQEWYKLHYMNIMAQVPLEKKINMS
YSAEELLVTCFFDGVSCDARNFTLFHHPMHGNCYTFNNRENETILSTSMG
GSEYGLQVILYINEEEYNPFLVSSTGAKVIIHRQDEYPFVEDVGTEIETA
MVTSIGMHLTESFKLSEPYSQCTEDGSDVPIRNIYNAAYSLQICLHSCFQ
TKMVEKCGCAQYSQPLPPAANYCNYQQHPNWMYCYYQLHRAFVQEELGCQ
SVCKEACSFKEWTLTTSLAQWPSVVSEKWLLPVLTWDQGRQVNKKLNKTD
LAKLLIFYKDLNQRSIMESPANSIEMLLSNFGGQLGLWMSCSVVCVIEII
EVFFIDFFSIIARRQWQKAKEWWAWKQAPPCPEAPRSPQGQDNPALDIDD
DLPTFNSALHLPPALGTQVPGTPPPKYNTLRLERAFSNQLTDTQMLDEL

γA splice variant nucleotide sequence (SEQ ID NO:21):

atggcacccggagagaagatcaaagccaaaatcaagaagaatctgcccgt
gacgggccctcaggcgccgaccattaaagagctgatgcggtggtactgcc
tcaacaccaacacccatggctgtcgccgcatcgtggtgtcccgcggccgt
ctgcgccgcctcctctggatcgggttcacactgactgccgtggccctcat
cctctggCagtgcgccctcctcgtcttctccttctatactgtctcagttt
ccatcaaagtccacttccggaagctggattttcctgcagtcaccatctgc
aacatcaacccctacaagtacagcaccgttcgccaccttctagctgactt
ggaacaggagaccagagaggccctgaagtccctgtatggctttccagagt
cccggaagcgccgagaggcggagtcctggaactccgtctcagagggaaag
cagcctagattctcccaccggattccgctgctgatctttgatcaggatga
gaagggcaaggccagggacttcttcacagggAggaagcggaaagtcggcg
gtagcatcattcacaaggcttcaaatgtcatgcacatcgagtccaagcaa
gtggtgggattccaactgtgctcaaatgacacctccgactgtgccaccta
caccttcagctcgggaatcaatgccattcaggagtggtataagctacact
acatgaacatcatggcacaggtgcctctggagaagaaaatcaacatgagc
tattctgctgaggagctgctggtgacctgcttctttgatggagtgtcctg
tgatgccaggaatttcacgcttttCcaccacccgatgcatgggaattgct
atactttcaacaacagagaaaatgagaccattctcagcacctccatgggg
ggcagcgaatatgggctgcaagtcattttgtacataaacgaagaggaata
caacccattcctcgtgtcctccactggagctaaggtgatcatccatcggc
aggatgagtatcccttcgtcgaagatgtgggaacagagattgagacagca
atggtcacctctataggaatgcacctgatctgcctCcattcatgcttcca
gacaaagatggtggagaaatgtgggtgtgcccagtacagccagcctctac
ctcctgcagccaactactgcaactaccagcagcaccccaactggatgtat
tgttactaccaactgcatcgagcctttgtccaggaagagctgggctgcca
gtctgtgtgcaaggaagcctgcagctttaaagagtggacactaaccacaa
gcctggcacaatggccatctgtggtttcggagaagtggttgctgcctgtt
ctcacttgggaccaaggccggcaagtaaacaaaaagctcaacaagacaga
cttgGccaaactcttgatattctacaaagacctgaaccagagatccatca
tggagagcccagccaacagtattgagatgcttctgtccaacttcggtggc
cagctgggcctgtggatgagctgctctgttgtctgcgtcatcgagatcat
cgaggtcttcttcattgacttcttctctatcattgcccgccgccagtggc
agaaagccaaggagtggtgggcctggaaacaggctcccccatgtccagaa
gctccccgtagcccacagggccaggacaatccagccctggatatagacga
tgacctacccactttcaactctgctttgcacctgcctccaGccctaggaa
cccaagtgcccggcacaccgccccccaaatacaataccttgcgcttggag
agggccttttccaaccagctcacagatacccagatgctGgatgagctctg
a

γA splice variant predicted protein sequence (SEQ ID NO:22):

MAPGEKIKAKIKKNLPVTGPQAPTIKELMRWYCLNTNTHGCRRIVVSRGR
LRRLLWIGFTLTAVALILWQCALLVFSFYTVSVSIKVHFRKLDFPAVTIC
NINPYKYSTVRHLLADLEQETREALKSLYGFPESRKRREAESWNSVSEGK
QPRFSHRIPLLIFDQDEKGKARDFFTGRKRKVGGSIIHKASNVMHIESKQ
VVGFQLCSNDTSDCATYTFSSGINAIQEWYKLHYMMNAQVPLEKKINMSY
SAEELLVTCFFDGVSCDARNFTLFHHPMHGNCYTFNNRENETILSTSMGG
SEYGLQVILYINEEEYNPFLVSSTGAKVIIHRQDEYPFVEDVGTEIETAM
VTSIGMHLICLHSCFQTKMVEKCGCAQYSQPLPPAANYCNYQQHPNWMYC
YYQLHRAFVQEELGCQSVCKEACSFKEWTLTTSLAQWPSVVSEKWLLPVL
TWDQGRQVNKKLNKTDLAKLLIFYKDLNQRSIMESPANSIEMLLSNFGGQ
LGLWMSCSVVCVIEIIEVFFIDFFSIIARRQWQKAKEWWAWKQAPPCPEA
PRSPQGQDNPALDIDDDLPTFNSALHLPPALGTQVPGTPPPKYNTLRLER
AFSNQLTDTQMLDEL

δ* splice variant nucleotide sequence (SEQ ID NO:23):

ACTCGGGAAGGCCACACAGCCAGTGACGAAGCTGTGATTCACACAGGCCT
GGGTGACTCCAGCATGGCTTTCCTCTCCAGGACGTCACCGGTGGCAGCTG
CTTCCTTCCAGAGCCGGCAGGAGGCCAGAGGCTCCATCCTGCTTCAGAGC
TGCCAGCTGCCCCCGCAatggctgagcaccgaagcatggacgggagaatg
gaagcagccacacgggggggctctcacctccagATCGCCTGGGCCTGTGG
CTCCCCAGAGGCCCTGCCACCTGAAGGGATGGCAGCACAGACCCACTCAG
CACAACGCTGCCTGCAAACAGGGCCAGgctgcagcccagacgccccccag
gccggggccaccatcagcaccaccaccaccacccaaggaggggcaccagg
aggggctggtggagctgcccgcctcgttccgggagctgctcaccttcttc
tgcaccaatgccaccatccacggcgccatccgcctggtctgctcccgcgg
gaaccgcctcaagacgacgtcctgggggctgctgtccctgggagccctgg
tcgcgctctgctggcagctggggctcctctttgagcgtcactggcaccgc
ccggtcctcatggccgtctctgtgcactcggagcgcaagctgctcccgct
ggtcaccctgtgtgacgggaacccacgtcggccgagtccggtcctccgcc
atctggagctgctggacgagtttgccagggagaacattgactccctgtac
aacgtcaacctcagcaaaggcagagccgccctctccgccactgtcccccg
ccacgagccccccttccacctggaccgggagatccgtctgcagaggctga
gccactcgggcagccgggtcagagtggggttcagactgtgcaacagcacg
ggcggcgactgcttttaccgaggctacacgtcaggcgtggcggctgtcca
ggactggtaccacttccactatgtggatatcctggccctgctgcccgcgg
catgggaggacagccacgggagccaggacggccacttcgtcctctcctgc
agttacgatggcctggactgccaggcccgacagttccggaccttccacca
ccccacctacggcagctgctacacggtcgatggcgtctggacagctcagc
gccccggcatcacccacggagtcggcctggtcctcagggttgagcagcag
cctcacctccctctgctgtccacgctggccggcatcagggtcatggttca
cggccgtaaccacacgcccttcctggggcaccacagcttcagcgtccggc
cagggacggaggccaccatcagcatccgagaggacgaggtgcaccggctc
gggagcccctacggccactgcaccgccggcggggaaggcgtggaggtgga
gctgctacacaacacctcctacaccaggcaggcctgcctggtgtcctgct
tccagcagctgatggtggagacctgctcctgtggctactacctccaccct
ctgccggcgggggctgagtactgcagctctgcccggcaccctgcctgggg
acactgcttctaccgcctctaccaggacctggagacccaccggctcccct
gtacctcccgctgccccaggccctgcagggagtctgcattcaagctctcc
actgggacctccaggtggccttccgccaagtcagctggatggactctggc
cacgctaggtgaacaggggctgccgcatcagagccacagacagaggagca
gcctggccaaaatcaacatcgtctaccaggagctcaactaccgctcagtg
gaggaggcgcccgtgtactcggtgccgcagctgctctccgccatgggcag
cctctacagcctgtggtttggggcctccgtcctctccctcctggagctcc
tggagctgctgctcgatgcttctgccctcaccctggtgctaggcggccgc
cggctccgcagggcgtggttctcctggcccagagccagccctgcctcagg
ggcgtccagcatcaagccagaggccagtcagatgcccccgcctgcaggcg
gcacgtcagatgacccggagcccagcgggcctcatctcccacgggtgatg
cttccaggggttctggcgggagtctcagccgaagagagctgggctgggcc
ccagccccttgagactctggacacctgaACCAGACCTGCCAGGGCTGTGC
GATCTCTTGGCCTGGTCCTTGCAGCTGTGGCAGCAGCAGGCTCCCCAGCG
GCCCAGGGTGGGCCAGACCAGCAGCCCAGGAAGCAGCACACGCGGCCGTG
GGGAGGCAGGCACCGGGCATGTCGGCGCCTCTGGTCAAACCACCTACACT
GCCTGGGGTGGGTCTCAAGGAGGCCCGGGGCGGAGGGGGGTTCCCGCGTG
CACACGAGTGCGGCTGGACGTGCCGACACGCGGTGATGTACCCATGCTCC
GTGTGTCTGTGTCTGCATGTCCACACGTCTGATGCACCTGTGTACGTGTG
TCAAGCCTAGCCACCTCAGCTGCAGGGAGGCAGAAGGCAAGGCAGGCCCC
ACGGACACACTTGGGCTGCTCTGAAATAAAGCTGTTGACTCCACCTG

δ* splice variant protein sequence (SEQ ID NO:24):

MAFLSRTSPVAAASFQSRQEARGSILLQSCQLPPQWLSTEAWTGEWKQPH
GGALTSRSPGPVAPQRPCHLKGWQHRPTQHNAACKQGQAAAQTPPRPGPP
SAPPPPPKEGHQEGLVELPASFRELLTFFCTNATIHGAIRLVCSRGNRLK
TTSWGLLSLGALVALCWQLGLLFERHWHRPVLMAVSVHSERKLLPLVTLC
DGNPRRPSPVLRHLELLDEFARENIDSLYNVNLSKGRAALSATVPRHEPP
FHLDREIRLQRLSHSGSRVRVGFRLCNSTGGDCFYRGYTSGVAAVQDWYH
FHYVDILALLPAAWEDSHGSQDGHFVLSCSYDGLDCQARQFRTFHHPTYG
SCYTVDGVWTAQRPGITHGVGLVLRVEQQPHLPLLSTLAGIRVMVHGRNH
TPFLGHHSFSVRPGTEATISIREDEVHRLGSPYGHCTAGGEGVEVELLHN
TSYTRQACLVSCFQQLMVETCSCGYYLHPLPAGAEYCSSARHPAWGHCFY
RLYQDLETHRLPCTSRCPRPCRESAFKLSTGTSRWPSAKSAGWTLATLGE
QGLPHQSHRQRSSLAKINIVYQELNYRSVEEAPVYSVPQLLSAMGSLYSL
WFGASVLSLLELLELLLDASALTLVLGGRRLRRAWFSWPRASPASGASSI
KPEASQMPPPAGGTSDDPEPSGPHLPRVMLPGVLAGVSAEESWAGPQPLE
TLDT

Reference kidney α1 nucleotide sequence (SEQ ID NO:1):

atggaggggaacaagctggaggagcaggactctagccctccacagtccac
tccagggctcatgaaggggaacaagcgtgaggagcaggggctgggccccg
aacctgcggcgccccagcagcccacggcggaggaggaggccctgatcgag
ttccaccgctcctaccgagagctcttcgagttcttctgcaacaacaccac
catccacggcgccatccgcctggtgtgctcccagcacaaccgcatgaaga
cggccttctgggcagtgctgtggctctgcacctttggcatgatgtactgg
caattcggcctgcttttcggagagtacttcagctaccccgtcagcctcaa
catcaacctcaactcggacaagctcgtcttccccgcagtgaccatctgca
ccctcaatccctacaggtacccggaaattaaagaggagctggaggagctg
gaccgcatcacagagcagacgctctttgacctgtacaaatacagctcctt
caccactctcgtggccggctcccgcagccgtcgcgacctgcgggggactc
tgccgcaccccttgcagcgcctgagggtcccgcccccgcctcacggggcc
cgtcgagcccgtagcgtggcctccagcttgcgggacaacaacccccaggt
ggactggaaggactggaagatcggcttccagctgtgcaaccagaacaaat
cggactgcttctaccagacatactcatcaggggtggatgcggtgagggag
tggtaccgcttccactacatcaacatcctgtcgaggctgccagagactct
gccatccctggaggaggacacgctgggcaacttcatcttcgcctgccgct
tcaaccaggtctcctgcaaccaggcgaattactctcacttccaccacccg
atgtatggaaactgctatactttcaatgacaagaacaactccaacctctg
gatgtcttccatgcctggaatcaacaacggtctgtccctgatgctgcgcg
cagagcagaatgacttcattcccctgctgtccacagtgactggggcccgg
gtaatggtgcacgggcaggatgaacctgcctttatggatgatggtggctt
taacttgcggcctggcgtggagacctccatcagcatgaggaaggaaaccc
tggacagacttgggggcgattatggcgactgcaccaagaatggcagtgat
gttcctgttgagaacctttacccttcaaagtacacacagcaggtgtgtat
tcactcctgcttccaggagagcatgatcaaggagtgtggctgtgcctaca
tcttctatccgcggccccagaacgtggagtactgtgactacagaaagcac
agttcctgggggtactgctactataagctccaggttgacttctcctcaga
ccacctgggctgtttcaccaagtgccggaagccatgcagcgtgaccagct
accagctctctgctggttactcacgatggccctcggtgacatcccaggaa
tgggtcttccagatgctatcgcgacagaacaattacaccgtcaacaacaa
gagaaatggagtggccaaagtcaacatcttcttcaaggagctgaactaca
aaaccaattctgagtctccctctgtcacgatggtcaccctcctgtccaac
ctgggcagccagtggagcctgtggttcggctcctcggtgttgtctgtggt
ggagatggctgagctcgtctttgacctgctggtcatcatgttcctcatgc
tgctccgaaggttccgaagccgatactggtctccaggccgagggggcagg
ggtgctcaggaggtagcctccaccctggcatcctcccctccttcccactt
ctgcccccaccccatgtctctgtccttgtcccagccaggccctgctccct
ctccagccttgacagcccctccccctgcctatgccaccctgggcccccgc
ccatctccagggggctctgcaggggccagttcctccacctgtcctctggg
ggggccctgagagggaaggagaggtttctcacaccaaggcagatgctcct
ctggtgggagggtgctggccctggcaagattgaaggatgtgcaggaattc

Predicted kidney α1 protein sequence (SEQ ID NO:2):

MEGNKLEEQDSSPPQSTPGLMKGNKREEQGLGPEPAAPQQPTAEEEALIE
FHRSYRELFEFFCNNTTIHGAIRLVCSQHNRMKTAFWAVLWLCTFGMMYW
QFGLLFGEYFSYPVSLNINLNSDKLVFPAVTICTLNPYRYPEIKEELEEL
DRITEQTLFDLYKYSSFTTLVAGSRSRRDLRGTLPHPLQRLRVPPPPHGA
RRARSVASSLRDNNPQVDWKDWKIGFQLCNQNKSDCFYQTYSSGVDAVRE
WYRFHYINILSRLPETLPSLEEDTLGNFIFACRFNQVSCNQANYSHFHHP
MYGNCYTFNDKNNSNLWMSSMPGINNGLSLMLRAEQNDFIPLLSTVTGAR
VMVHGQDEPAFMDDGGFNLRPGVETSISMRKETLDRLGGDYGDCTKNGSD
VPVENLYPSKYTQQVCIHSCFQESMIKECGCAYIFYPRPQNVEYCDYRKH
SSWGYCYYKLQVDFSSDHLGCFTKCRKPCSVTSYQLSAGYSRWPSVTSQE
WVFQMLSRQNNYTVNNKRNGVAKVNIFFKELNYKTNSESPSVTMVTLLSN
LGSQWSLWFGSSVLSVVEMAELVFDLLVIMFLMLLRRFRSRYWSPGRGGR
GAQEVASTLASSPPSHFCPHPMSLSLSQPGPAPSPALTAPPPAYATLGPR
PSPGGSAGASSSTCPLGGP

α1A splice variant nucleotide sequence ((SEQ ID NO:3):

atggaggggaacaagctggaggagcaggactctagccctccacagtccac
tccagggctcatgaaggggaacaagcgtgaggagcaggggctgggccccg
aacctgcggcgccccagcagcccacggcggaggaggaggccctgatcgag
ttccaccgctcctaccgagagctcttcgagttcttctgcaacaacaccac
catccacggcgccatccgcctggtgtgctcccagcacaaccgcatgaaga
cggccttctgggcagtgctgtggctctgcacctttggcatgatgtactgg
caattcggcctgcttttcggagagtacttcagctaccccgtcagcctcaa
catcaacctcaactcggacaagctcgtcttccccgcagtgaccatctgca
ccctcaatccctacaggtacccggaaattaaagaggagctggaggagctg
gaccgcatcacagagcagacgctctttgacctgtacaaatacagctcctt
caccactctcgtggccggctcccgcagccgtcgcgacctgcgggggactc
tgccgcaccccttgcagcgcctgagggtcccgcccccgcctcacggggcc
cgtcgagcccgtagcgtggcctccagcttgcgggacaacaacccccaggt
ggactggaaggactggaagatcggcttccagctgtgcaaccagaacaaat
cggactgcttctaccagacatactcatcaggggtggatgcggtgagggag
tggtaccgcttccactacatcaacatcctgtcgaggctgccagagactct
gccatccctggaggaggacacgctgggcaacttcatcttcgcctgccgct
tcaaccaggtctcctgcaaccaggcgaattactctcacttccaccacccg
atgtatggaaactgctatactttcaatgacaagaacaactccaacctctg
gatgtcttccatgcctggaatcaacaacgtgactggggcccgggtaatgg
tgcacgggcaggatgaacctgcctttatggatgatggtggctttaacttg
cggcctggcgtggagacctccatcagcatgaggaaggaaaccctggacag
acttgggggcgattatggcgactgcaccaagaatggcagtgatgttcctg
ttgagaacctttacccttcaaagtacacacagcaggtgtgtattcactcc
tgcttccaggagagcatgatcaaggagtgtggctgtgcctacatcttcta
tccgcggccccagaacgtggagtactgtgactacagaaagcacagttcct
gggggtactgctactataagctccaggttgacttctcctcagaccacctg
ggctgtttcaccaagtgccggaagccatgcagcgtgaccagctaccagct
ctctgctggttactcacgatggccctcggtgacatcccaggaatgggtct
tccagatgctatcgcgacagaacaattacaccgtcaacaacaagagaaat
ggagtggccaaagtcaacatcttcttcaaggagctgaactacaaaaccaa
ttctgagtctccctctgtcacgatggtcaccctcctgtccaacctgggca
gccagtggagcctgtggttcggctcctcggtgttgtctgtggtggagatg
gctgagctcgtctttgacctgctggtcatcatgttcctcatgctgctccg
aaggttccgaagccgatactggtctccaggccgagggggcaggggtgctc
aggaggtagcctccaccctggcatcctcccctccttcccacttctgcccc
caccccatgtctctgtccttgtcccagccaggccctgctccctctccagc
cttgacagcccctccccctgcctatgccaccctgggcccccgcccatctc
cagggggctctgcaggggccagttcctccacctgtcctctgggggggccc
tga

α1A splice variant predicted protein sequence (SEQ ID NO:4):

MEGNKLEEQDSSPPQSTPGLMKGNKREEQGLGPEPAAPQQPTAEEEALIE
FHRSYRELFEFFCNNTTIHGAIRLVCSQHNRMKTAFWAVLWLCTFGMMYW
QFGLLFGEYFSYPVSLNINLNSDKLVFPAVTICTLNPYRYPEIKEELEEL
DRITEQTLFDLYKYSSFTTLVAGSRSRRDLRGTLPHPLQRLRVPPPPHGA
RRARSVASSLRDNNPQVDWKDWKIGFQLCNQNKSDCFYQTYSSGVDAVRE
WYRFHYINILSRLPETLPSLEEDTLGNFIFACRFNQVSCNQANYSHFHHP
MYGNCYTFNDKNNSNLWMSSMPGINNVTGARVMVHGQDEPAFMDDGGFNL
RPGVETSISMRKETLDRLGGDYGDCTKNGSDVPVENLYPSKYTQQVCIHS
CFQESMIKECGCAYIFYPRPQNVEYCDYRKHSSWGYCYYKLQVDFSSDHL
GCFTKCRKPCSVTSYQLSAGYSRWPSVTSQEWVFQMLSRQNNYTVNNKRN
GVAKVNIFFKELNYKTNSESPSVTMVTLLSNLGSQWSLWFGSSVLSVVEM
AELVFDLLVIMFLMLLRRFRSRYWSPGRGGRGAQEVASTLASSPPSHFCP
HPMSLSLSQPGPAPSPALTAPPPAYATLGPRPSPGGSAGASSSTCPLGGP

α2 reference nucleotide sequence (SEQ ID NO:5):

atgggcatggccaggggcagcctcactcgggttccaggggtgatgggaga
gggcactcagggcccagagctcagccttgaccctgacccttgctctcccc
aatccactccggggctcatgaaggggaacaagctggaggagcaggaccct
agacctctgcagcccataccaggtctcatggaggggaacaagctggagga
gcaggactctagccctccacagtccactccagggctcatgaaggggaaca
agcgtgaggagcaggggctgggccccgaacctgcggcgccccagcagccc
acggcggaggaggaggccctgatcgagttccaccgctcctaccgagagct
cttcgagttcttctgcaacaacaccaccatccacggcgccatccgcctgg
tgtgctcccagcacaaccgcatgaagacggccttctgggcagtgctgtgg
ctctgcacctttggcatgatgtactggcaattcggcctgcttttcggaga
gtacttcagctaccccgtcagcctcaacatcaacctcaactcggacaagc
tcgtcttccccgcagtgaccatctgcaccctcaatccctacaggtacccg
gaaattaaagaggagctggaggagctggaccgcatcacagagcagacgct
ctttgacctgtacaaatacagctccttcaccactctcgtggccggctccc
gcagccgtcgcgacctgcgggggactctgccgcaccccttgcagcgcctg
agggtcccgcccccgcctcacggggcccgtcgagcccgtagcgtggcctc
cagcttgcgggacaacaacccccaggtggactggaaggactggaagatcg
gcttccagctgtgcaaccagaacaaatcggactgcttctaccagacatac
tcatcaggggtggatgcggtgagggagtggtaccgcttccactacatcaa
catcctgtcgaggctgccagagactctgccatccctggaggaggacacgc
tgggcaacttcatcttcgcctgccgcttcaaccaggtctcctgcaaccag
gcgaattactctcacttccaccacccgatgtatggaaactgctatacttt
caatgacaagaacaactccaacctctggatgtcttccatgcctggaatca
acaacggtctgtccctgatgctgcgcgcagagcagaatgacttcattccc
ctgctgtccacagtgactggggcccgggtaatggtgcacgggcaggatga
acctgcctttatggatgatggtggctttaacttgcggcctggcgtggaga
cctccatcagcatgaggaaggaaaccctggacagacttgggggcgattat
ggcgactgcaccaagaatggcagtgatgttcctgttgagaacctttaccc
ttcaaagtacacacagcaggtgtgtattcactcctgcttccaggagagca
tgatcaaggagtgtggctgtgcctacatcttctatccgcggccccagaac
gtggagtactgtgactacagaaagcacagttcctgggggtactgctacta
taagctccaggttgacttctcctcagaccacctgggctgtttcaccaagt
gccggaagccatgcagcgtgaccagctaccagctctctgctggttactca
cgatggccctcggtgacatcccaggaatgggtcttccagatgctatcgcg
acagaacaattacaccgtcaacaacaagagaaatggagtggccaaagtca
acatcttcttcaaggagctgaactacaaaaccaattctgagtctccctct
gtcacgatggtcaccctcctgtccaacctgggcagccagtggagcctgtg
gttcggctcctcggtgttgtctgtggtggagatggctgagctcgtctttg
acctgctggtcatcatgttcctcatgctgctccgaaggttccgaagccga
tactggtctccaggccgagggggcaggggtgctcaggaggtagcctccac
cctggcatcctcccctccttcccacttctgcccccaccccatgtctctgt
ccttgtcccagccaggccctgctccctctccagccttgacagcccctccc
cctgcctatgccaccctgggcccccgcccatctccagggggctctgcagg
ggccagttcctccacctgtcctctgggggggccctga

α2 reference predicted protein sequence (SEQ ID NO:6):

MGMARGSLTRVPGVMGEGTQGPELSLDPDPCSPQSTPGLMKGNKLEEQDP
RPLQPIPGLMEGNKLEEQDSSPPQSTPGLMKGNKREEQGLGPEPAAPQQP
TAEEEALIEFHRSYRELFEFFCNNTTIHGAIRLVCSQHNRMKTAFWAVLW
LCTFGMMYWQFGLLFGEYFSYPVSLNINLNSDKLVFPAVTICTLNPYRYP
EIKEELEELDRITEQTLFDLYKYSSFTTLVAGSRSRRDLRGTLPHPLQRL
RVPPPPHGARRARSVASSLRDNNPQVDWKDWKIGFQLCNQNKSDCFYQTY
SSGVDAVREWYRFHYINILSRLPETLPSLEEDTLGNFIFACRFNQVSCNQ
ANYSHFHHPMYGNCYTFNDKNNSNLWMSSMPGINNGLSLMLRAEQNDFIP
LLSTVTGARVMVHGQDEPAFMDDGGFNLRPGVETSISMRKETLDRLGGDY
GDCTKNGSDVPVENLYPSKYTQQVCIHSCFQESMIKECGCAYIFYPRPQN
VEYCDYRKHSSWGYCYYKLQVDFSSDHLGCFTKCRKPCSVTSYQLSAGYS
RWPSVTSQEWVFQMLSRQNNYTVNNKRNGVAKVNIFFKELNYKTNSESPS
VTMVTLLSNLGSQWSLWFGSSVLSVVEMAELVFDLLVIMFLMLLRRFRSR
YWSPGRGGRGAQEVASTLASSPPSHFCPHPMSLSLSQPGPAPSPALTAPP
PAYATLGPRPSPGGSAGASSSTCPLGGP

α2A splice variant nucleotide sequence (SEQ ID NO:7):

atgggcatggccaggggcagcctcactcgggttccaggggtgatgggaga
gggcactcagggcccagagctcagccttgaccctgacccttgctctcccc
aatccactccggggctcatgaaggggaacaagctggaggagcaggaccct
agacctctgcagcccataccaggtctcatggaggggaacaagctggagga
gcaggactctagccctccacagtccactccagggctcatgaaggggaaca
agcgtgaggagcaggggctgggccccgaacctgcggcgccccagcagccc
acggcggaggaggaggccctgatcgagttccaccgctcctaccgagagct
cttcgagttcttctgcaacaacaccaccatccacggcgccatccgcctgg
tgtgctcccagcacaaccgcatgaagacggccttctgggcagtgctgtgg
ctctgcacctttggcatgatgtactggcaattcggcctgcttttcggaga
gtacttcagctaccccgtcagcctcaacatcaacctcaactcggacaagc
tcgtcttccccgcagtgaccatctgcaccctcaatccctacaggtacccg
gaaattaaagaggagctggaggagctggaccgcatcacagagcagacgct
ctttgacctgtacaaatacagctccttcaccactctcgtggccggctccc
gcagccgtcgcgacctgcgggggactctgccgcaccccttgcagcgcctg
agggtcccgcccccgcctcacggggcccgtcgagcccgtagcgtggcctc
cagcttgcgggacaacaacccccaggtggactggaaggactggaagatcg
gcttccagctgtgcaaccagaacaaatcggactgcttctaccagacatac
tcatcaggggtggatgcggtgagggagtggtaccgcttccactacatcaa
catcctgtcgaggctgccagagactctgccatccctggaggaggacacgc
tgggcaacttcatcttcgcctgccgcttcaaccaggtctcctgcaaccag
gcgaattactctcacttccaccacccgatgtatggaaactgctatacttt
caatgacaagaacaactccaacctctggatgtcttccatgcctggaatca
acaacgtgactggggcccgggtaatggtgcacgggcaggatgaacctgcc
tttatggatgatggtggctttaacttgcggcctggcgtggagacctccat
cagcatgaggaaggaaaccctggacagacttgggggcgattatggcgact
gcaccaagaatggcagtgatgttcctgttgagaacctttacccttcaaag
tacacacagcaggtgtgtattcactcctgcttccaggagagcatgatcaa
ggagtgtggctgtgcctacatcttctatccgcggccccagaacgtggagt
actgtgactacagaaagcacagttcctgggggtactgctactataagctc
caggttgacttctcctcagaccacctgggctgtttcaccaagtgccggaa
gccatgcagcgtgaccagctaccagctctctgctggttactcacgatggc
cctcggtgacatcccaggaatgggtcttccagatgctatcgcgacagaac
aattacaccgtcaacaacaagagaaatggagtggccaaagtcaacatctt
cttcaaggagctgaactacaaaaccaattctgagtctccctctgtcacga
tggtcaccctcctgtccaacctgggcagccagtggagcctgtggttcggc
tcctcggtgttgtctgtggtggagatggctgagctcgtctttgacctgct
ggtcatcatgttcctcatgctgctccgaaggttccgaagccgatactggt
ctccaggccgagggggcaggggtgctcaggaggtagcctccaccctggca
tcctcccctccttcccacttctgcccccaccccatgtctctgtccttgtc
ccagccaggccctgctccctctccagccttgacagcccctccccctgcct
atgccaccctgggcccccgcccatctccagggggctctgcaggggccagt
tcctccacctgtcctctgggggggccctga

α2A splice variant predicted protein sequence (SEQ ID NO:8):

MGMARGSLTRVPGVMGEGTQGPELSLDPDPCSPQSTPGLMKGNKLEEQDP
RPLQPIPGLMEGNKLEEQDSSPPQSTPGLMKGNKREEQGLGPEPAAPQQP
TAEEEALIEFHRSYRELFEFFCNNTTIHGAIRLVCSQHNRMKTAFWAVLW
LCTFGMMYWQFGLLFGEYFSYPVSLNINLNSDKLVFPAVTICTLNPYRYP
EIKEELEELDRITEQTLFDLYKYSSFTTLVAGSRSRRDLRGTLPHPLQRL
RVPPPPHGARRARSVASSLRDNNPQVDWKDWKIGFQLCNQNKSDCFYQTY
SSGVDAVREWYRFHYINILRLPETLPSLEEDTLGNFIFACRFNQVSCNQA
NYSHFHHPMYGNCYTFNDKNNSNLWMSSMPGINNVTGARVMVHGQDEPAF
MDDGGFNLRPGVETSISMRKETLDRLGGDYGDCTKNGSDVPVENLYPSKY
TQQVCIHSCFQESMIKECGCAYIFYPRPQNVEYCDYRKHSSWGYCYYKLQ
VDFSSDHLGCFTKCRKPCSVTSYQLSAGYSRWPSVTSQEWVFQMLSRQNN
YTVNNKRNGVAKVNIFFKELNYKTNSESPSVTMVTLLSNLGSQWSLWFGS
SVLSVVEMAELVFDLLVIMFLMLLRRFRSRYWSPGRGGRGAQEVASTLAS
SPPSHFCPHPMSLSLSQPGPAPSPALTAPPPAYATLGPRPSPGGSAGASS
STCPLGGP

β ENaC kidney reference nucleotide sequence (SEQ ID NO:9):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgggttcctgctcaccctgctcttcgccgccctcgtctgctggcagtgg
ggcatcttcatcaggacctacttgagctgggaggtcagcgtctccctctc
cgtaggcttcaagaccatggacttccccgccgtcaccatctgcaatgcta
gccccttcaagtattccaaaatcaagcatttgctgaaggacctggatgag
ctgatggaagctgtcctggagagaatcctggctcctgagctaagccatgc
caatgccaccaggaacctgaacttctccatctggaaccacacacccctgg
tccttattgatgaacggaacccccaccaccccatggtccttgatctcttt
ggagacaaccacaatggcttaacaagcagctcagcatcagaaaagatctg
taatgcccacgggtgcaaaatggccatgagactatgtagcctcaacagga
cccagtgtaccttccggaacttcaccagtgctacccaggcattgacagag
tggtacatcctgcaggccaccaacatctttgcacaggtgccacagcagga
gctagtagagatgagctaccccggcgagcagatgatcctggcctgcctat
tcggagctgagccctgcaactaccggaacttcacgtccatcttctaccct
cactatggcaactgttacatcttcaactggggcatgacagagaaggcact
tccttcggccaaccctggaactgaattcggcctgaagttgatcctggaca
taggccaggaagactacgtccccttccttgcgtccacggccggggtcagg
ctgatgcttcacgagcagaggtcataccccttcatcagagatgagggcat
ctacGccatgtcggggacagagacgtccatcggggtactcgtggacaagc
ttcagcgcatgggggagccctacagcccgtgcaccgtgaatggttctgag
gtccccgtccaaaacttctacagtgactacaacacgacctactccatcca
ggcctgtcttcgctcctgcttccaagaccacatgatccgtaactgcaact
gtggccactacctgtacccactGccccgtggggagaaatactgcaacaac
cgggacttcccagactgggcccattgctactcagatctacagatgagcgt
ggcgcagagagagacctgcattggcatgtgcaaggagtcctgcaatgaca
cccagtacaagatgaccatctccatggctgactggccttctgaggcctcc
gaggactggattttccacgtcttgtctcaggagcgggaccaaagcaccaa
tatcaccctgagcaggaagggaattgtcaagctcaacatctActtccaag
aatttaactatcgcaccattgaagaatcagcagccaataacatcgtctgg
ctgctctcgaatctgggtggccagtttggcttctggatggggggctctgt
gctgtgcctcatcgagtttggggagatcatcatcgactttgtgtggatca
ccatcatcaagctggtggccttggccaagagcctacggcagcggcgagcc
caagccagCtacgctggcccaccgcccaccgtggccgagctggtggaggc
ccacaccaactttggcttccagcctgacacggccccccgcagccccaaca
ctgggccctaccccagtgagcaggccctgcccatcccaggcaccccgccc
cccaactatgactccctgcgtctgcagccgctggacgtcatcgagtctga
cagtgagggtgatgccatctaa

β ENaC kidney reference predicted protein sequence (SEQ ID NO:10):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAQVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGVLVDK
LQRMGEPYSPCTVNGSEVPVQNFYSDYNTTYSIQACLRSCFQDHMIRNCN
CGHYLYPLPRGEKYCNNRDFPDWAHCYSDLQMSVAQRETCIGMCKESCND
TQYKMTISMADWPSEASEDWIFHVLSQERDQSTNITLSRKGIVKLNIYFQ
EFNYRTIEESAANNIVWLLSNLGGQFGFWMGGSVLCLIEFGEIIIDFVWI
TIIKLVALAKSLRQRRAQASYAGPPPTVAELVEAHTNFGFQPDTAPRSPN
TGPYPSEQALPIPGTPPPNYDSLRLQPLDVIESDSEGDAI

βA splice variant nucleotide sequence (SEQ ID NO:11):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcggggtactcgtggcctgt
cttcgctcctgcttccaagaccacatgatccgtaactgcaactgtggcca
ctacctgtacccactGccccgtggggagaaatactgcaacaaccgggact
tcccagactgggcccattgctactcagatctacagatgagcgtggcgcag
agagagacctgcattggcatgtgcaaggagtcctgcaatgacacccagta
caagatgaccatctccatggctgactggccttctgaggcctccgaggact
ggattttccacgtcttgtctcaggagcgggaccaaagcaccaatatcacc
ctgagcaggaagggaattgtcaagctcaacatctActtccaagaatttaa
ctatcgcaccattgaagaatcagcagccaataacatcgtctggctgctct
cgaatctgggtggccagtttggcttctggatggggggctctgtgctgtgc
ctcatcgagtttggggagatcatcatcgactttgtgtggatcaccatcat
caagctggtggccttggccaagagcctacggcagcggcgagcccaagcca
gCtacgctggcccaccgcccaccgtggccgagctggtggaggcccacacc
aactttggcttccagcctgacacggccccccgcagccccaacactgggcc
ctaccccagtgagcaggccctgcccatcccaggcaccccgccccccaact
atgactccctgcgtctgcagccgctggacgtcatcgagtctgacagtgag
ggtgatgccatctaa

βA splice variant predicted protein sequence (SEQ ID NO:12):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKYSKIKHLLKDLDELMEAVLERILAPELSHANATRNLNFSIWNHTPL
VLIDERNPHHPMVLDLFGDNHNGLTSSSASEKICNAHGCKMAMRLCSLNR
TQCTFRNFTSATQALTEWYILQATNIFAWVPQQELVEMSYPGEQMILACL
FGAEPCNYRNFTSIFYPHYGNCYIFNWGMTEKALPSANPGTEFGLKLILD
IGQEDYVPFLASTAGVRLMLHEQRSYPFIRDEGIYAMSGTETSIGVLVAC
LRSCFQDHMIRNCNCGHYLYPLPRGEKYCNNRDFPDWAHCYSDLQMSVAQ
RETCIGMCKESCNTQYKMTISMADWPSEASEDWIFHVLSQERDQSTNITL
SRKGIVKLNIYFQEFNYRTIEESAANNIVWLLSNLGGQFGFWMGGSVLCL
IEFGEIIIDFVWITIIKLVALAKSLRQRRAQASYAGPPPTVAELVEAHTN
FGFQPTAPRSPNTGPYPSEQALPIPGTPPPNYDSLRLQPLDVIESDSEGD
AI

βB splice variant nucleotide sequence (#13):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagaacttcacgtccatcttctaccctcactatggcaactg
ttacatcttcaactggggcatgacagagaaggcacttccttcggccaacc
ctggaactgaattcggcctgaagttgatcctggacataggccaggaagac
tacgtccccttccttgcgtccacggccggggtcaggctgatgcttcacga
gcagaggtcataccccttcatcagagatgagggcatctacGccatgtcgg
ggacagagacgtccatcggggtactcgtggacaagcttcagcgcatgggg
gagccctacagcccgtgcaccgtgaatggttctgaggtccccgtccaaaa
cttctacagtgactacaacacgacctactccatccaggcctgtcttcgct
cctgcttccaagaccacatgatccgtaactgcaactgtggccactacctg
tacccactGccccgtggggagaaatactgcaacaaccgggacttcccaga
ctgggcccattgctactcagatctacagatgagcgtggcgcagagagaga
cctgcattggcatgtgcaaggagtcctgcaatgacacccagtacaagatg
accatctccatggctgactggccttctgaggcctccgaggactggatttt
ccacgtcttgtctcaggagcgggaccaaagcaccaatatcaccctgagca
ggaagggaattgtcaagctcaacatctActtccaagaatttaactatcgc
accattgaagaatcagcagccaataacatcgtctggctgctctcgaatct
gggtggccagtttggcttctggatggggggctctgtgctgtgcctcatcg
agtttggggagatcatcatcgactttgtgtggatcaccatcatcaagctg
gtggccttggccaagagcctacggcagcggcgagcccaagccagCtacgc
tggcccaccgcccaccgtggccgagctggtggaggcccacaccaactttg
gcttccagcctgacacggccccccgcagccccaacactgggccctacccc
agtgagcaggccctgcccatcccaggcaccccgccccccaactatgactc
cctgcgtctgcagccgctggacgtcatcgagtctgacagtgagggtgatg
ccatctaa

βB splice variant predicted protein sequence (SEQ ID NO:14):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICNA
SPFKNFTSIFYPHYGNCYIFNWGMTEKALPSANMPGTEFGLKLILDIGQE
DYVPFLASTAGVRLMLHEQRSYPFIRDEGIYAMSGTETSIGVLVDKLQRM
GEPYSPCTVNGSEVPVQNFYSDYNTTYSIQACLRSCFQDHMIRNCNCGHY
LYPLPRGEKYCNNRDFPDWAHCYSDLQMSVAQRETCIGMCKESCNDTQYK
MTISMADWPSEASEDWIFHVLSQERDQSTNITLSRKGIVKLNIYFQEFNY
RTIEESAANNIVWLLSNLGGQFGFWMGGSVLCLIEFGEIIIDFVWITIIK
LVALAKSLRQRRAQASYAGPPPTVAELVEAHTNFGFQPDTAPRSPNTGPY
PSEQALPIPGTPPPNYDSLRLQPLDVIESDSEGDAI

β* splice variant nucleotide sequence (SEQ ID NO:15):

atgcacgtgaagaagtacctGctgaagggcctgcatcggctgcagaaggg
ccccggctacacgtacaaggagctgctggtgtggtactgcgacaacacca
acacccacggccccaagcgcatcatctgtgaggggcccaagaagaaagcc
atgtggttcctgctcaccctgctcttcgccgccctcgtctgctggcagtg
gggcatcttcatcaggacctacttgagctgggaggtcagcgtctccctct
ccgtaggcttcaagaccatggacttccccgccgtcaccatctgcaatgct
agccccttcaagtattccaaaatcaagcatttgctgaaggacctggatga
gctgatggaagctgtcctggagagaatcctggctcctgagctaagccatg
ccaatgccaccaggaacctgaacttctccatctggaaccacacacccctg
gtccttattgatgaacggaacccccaccaccccatggtccttgatctctt
tggagacaaccacaatggcttaacaagcagctcagcatcagaaaagatct
gtaatgcccacgggtgcaaaatggccatgagactatgtagcctcaacagg
acccagtgtaccttccggaacttcaccagtgctacccaggcattgacaga
gtggtacatcctgcaggccaccaacatctttgcacaggtgccacagcagg
agctagtagagatgagctaccccggcgagcagatgatcctggcctgccta
ttcggagctgagccctgcaactaccggaacttcacgtccatcttctaccc
tcactatggcaactgttacatcttcaactggggcatgacagagaaggcac
ttccttcggccaaccctggaactgaattcggcctgaagttgatcctggac
ataggccaggaagactacgtccccttccttgcgtccacggccggggtcag
gctgatgcttcacgagcagaggtcataccccttcatcagagatgagggca
tctacGccatgtcggggacagagacgtccatcgggGACaagcttcagcgc
atgggggagccctacagcccgtgcaccgtgaatggttctgaggtccccgt
ccaaaacttctacagtgactacaacacgacctactccatccaggcctgtc
ttcgctcctgcttccaagaccacatgatccgtaactgcaactgtggccac
tacctgtacccactGccccgtggggagaaatactgcaacaaccgggactt
cccagactgggcccattgctactcagatctacagatgagcgtggcgcaga
gagagacctgcattggcatgtgcaaggagtcctgcaatgacacccagtac
aagatgaccatctccatggctgactggccttctgaggcctccgaggactg
gattttccacgtcttgtctcaggagcgggaccaaagcaccaatatcaccc
tgagcaggaagggaattgtcaagctcaacatctActtccaagaatttaac
tatcgcaccattgaagaatcagcagccaataacatcgtctggctgctctc
gaatctgggtggccagtttggcttctggatggggggctctgtgctgtgcc
tcatcgagtttggggagatcatcatcgactttgtgtggatcaccatcatc
aagctggtggccttggccaagagcctacggcagcggcgagcccaagccag
Ctacgctggcccaccgcccaccgtggccgagctggtggaggcccacacca
actttggcttccagcctgacacggccccccgcagccccaacactgggccc
taccccagtgagcaggccctgcccatcccaggcaccccgccccccaacta
tgactccctgcgtctgcagccgctggacgtcatcgagtctgacagtgagg
gtgatgccatctaa

β* splice variant predicted protein sequence (SEQ ID NO:16):

MHVKKYLLKGLHRLQKGPGYTYKELLVWYCDNTNTHGPKRIICEGPKKKA
MWFLLTLLFAALVCWQWGIFIRTYLSWEVSVSLSVGFKTMDFPAVTICAS
PFKYSKIKHLLKDLDELMEAVLERILAPELSHANATRNLNFSIWNHTPLV
LIDERNPHHPMVLDLFGDNHNGLTSSSASEKICNAHGCKMAMRLCSLNRT
QCTFRNFTSATQALTEWYILQATNIFAWVPQQELVEMSYPGEQMILACLF
GAEPCNYRNFTSIFYPHYGNCYIFNWGMTEKALPSANPGTEFGLKLILDI
GQEDYVPFLASTAGVRLMLHEQRSYPFIRDEGIYAMSGTETSIGdKLQRM
GEPYSPCTVNGSEVPVQNFYSDYNTTYSIQACLRSCFQDHMIRNCNCGHY
LYPLPRGEKYCNNRDFPDWAHCYSDLQMSVAQRETCIGMCKESCNTQYKM
TISMADWPSEASEDWIFHVLSQERDQSTNITLSRKGIVKLNIYFQEFNYR
TIEESAANNIVWLLSNLGGQFGFWMGGSVLCLIEFGEIIIDFVWITIIKL
VALAKSLRQRRAQASYAGPPPTVAELVEAHTNFGFQPDTAPRSPNTGPYP
SEQALPIPGTPPPNYDSLRLQPLDVIESDSEGDAI

γ ENaC kidney reference nucleotide sequence (SEQ ID NO:17):

atggcacccggagagaagatcaaagccaaaatcaagaagaatctgcccgt
gacgggccctcaggcgccgaccattaaagagctgatgcggtggtactgcc
tcaacaccaacacccatggctgtcgccgcatcgtggtgtcccgcggccgt
ctgcgccgcctcctctggatcgggttcacactgactgccgtggccctcat
cctctggcagtgcgccctcctcgtcttctccttctatactgtctcagttt
ccatcaaagtccacttccggaagctggattttcctgcagtcaccatctgc
aacatcaacccctacaagtacagcaccgttcgccaccttctagctgactt
ggaacaggagaccagagaggccctgaagtccctgtatggctttccagagt
cccggaagcgccgagaggcggagtcctggaactccgtctcagagggaaag
cagcctagattctcccaccggattccgctgctgatctttgatcaggatga
gaagggcaaggccagggacttcttcacagggAggaagcggaaagtcggcg
gtagcatcattcacaaggcttcaaatgtcatgcacatcgagtccaagcaa
gtggtgggattccaactgtgctcaaatgacacctccgactgtgccaccta
caccttcagctcgggaatcaatgccattcaggagtggtataagctacact
acatgaacatcatggcacaggtgcctctggagaagaaaatcaacatgagc
tattctgctgaggagctgctggtgacctgcttctttgatggagtgtcctg
tgatgccaggaatttcacgcttttCcaccacccgatgcatgggaattgct
atactttcaacaacagagaaaatgagaccattctcagcacctccatgggg
ggcagcgaatatgggctgcaagtcattttgtacataaacgaagaggaata
caacccattcctcgtgtcctccactggagctaaggtgatcatccatcggc
aggatgagtatcccttcgtcgaagatgtgggaacagagattgagacagca
atggtcacctctataggaatgcacctgacagagtccttcaagctgagtga
gccctacagtcagtgcacggaggacgggagtgacgtgccaatcaggaaca
tctacaacgctgcctactcgctccagatctgccttcattcatgcttccag
acaaagatggtggagaaatgtgggtgtgcccagtacagccagcctctacc
tcctgcagccaactactgcaactaccagcagcaccccaactggatgtatt
gttactaccaactgcatcgagcctttgtccaggaagagctgggctgccag
tctgtgtgcaaggaagcctgcagctttaaagagtggacactaaccacaag
cctggcacaatggccatctgtggtttcggagaagtggttgctgcctgttc
tcacttgggaccaaggccggcaagtaaacaaaaagctcaacaagacagac
ttgGccaaactcttgatattctacaaagacctgaaccagagatccatcat
ggagagcccagccaacagtattgagatgcttctgtccaacttcggtggcc
agctgggcctgtggatgagctgctctgttgtctgcgtcatcgagatcatc
gaggtcttcttcattgacttcttctctatcattgcccgccgccagtggca
gaaagccaaggagtggtgggcctggaaacaggctcccccatgtccagaag
ctccccgtagcccacagggccaggacaatccagccctggatatagacgat
gacctacccactttcaactctgctttgcacctgcctccaGccctaggaac
ccaagtgcccggcacaccgccccccaaatacaataccttgcgcttggaga
gggccttttccaaccagctcacagatacccagatgctAgatgagctctga

γ ENaC kidney reference predicted protein sequence (SEQ ID NO:18):

MAPGEKIKAKIKKNLPVTGPQAPTIKELMRWYCLNTNTHGCRRIVVSRGR
LRRLLWIGFTLTAVALILWQCALLVFSFYTVSVSIKVHFRKLDFPAVTIC
NINPYKYSTVRHLLADLEQETREALKSLYGFPESRKRREAESWNSVSEGK
QPRFSHRIPLLIFDQDEKGKARDFFTGRKRKVGGSIIHKASNVMHIESKQ
VVGFQLCSNDTSDCATYTFSSGINAIQEWYKLHYMNIMAQVPLEKKINMS
YSAEELLVTCFFDGVSCDARNFTLFHHPMHGNCYTFNNRENETILSTSMG
GSEYGLQVILYINEEEYNPFLVSSTGAKVIIHRQDEYPFVEDVGTEIETA
MVTSIGMHLTESFKLSEPYSQCTEDGSDVPIRNIYNAAYSLQICLHSCFQ
TKMVEKCGCAQYSQPLPPAANYCNYQQNPNWMYCYYQLHRAFVQEELGCQ
SVCKEACSFKEWTLTTSLAQWPSVVSEKWLLPVLTWDQGRQVNKKLNKTD
LAKLLIFYKDLNQRSIMESPANSIEMLLSNFGGQLGLWMSCSVVCVIEII
EVFFIDFFSIIARRQWQKAKEWWAWKQAPPCPEAPRSPQGQDNPALDIDD
DLPTFNSALHLPPALGTQVPGTPPPKYNTLRLERAFSNQLTDTQMLDEL

γA splice variant nucleotide sequence (SEQ ID NO:19):

atggcacccggagagaagatcaaagccaaaatcaagaagaatctgcccgt
gacgggccctcaggcgccgaccattaaagagctgatgcggtggtactgcc
tcaacaccaacacccatggctgtcgccgcatcgtggtgtcccgcggccgt
ctgcgccgcctcctctggatcgggttcacactgactgccgtggccctcat
cctctggCagtgcgccctcctcgtcttctccttctatactgtctcagttt
ccatcaaagtccacttccggaagctggattttcctgcagtcaccatctgc
aacatcaacccctacaagtacagcaccgttcgccaccttctagctgactt
ggaacaggagaccagagaggccctgaagtccctgtatggctttccagagt
cccggaagcgccgagaggcggagtcctggaactccgtctcagagggaaag
cagcctagattctcccaccggattccgctgctgatctttgatcaggatga
gaagggcaaggccagggacttcttcacagggAggaagcggaaagtcggcg
gtagcatcattcacaaggcttcaaatgtcatgcacatcgagtccaagcaa
gtggtgggattccaactgtgctcaaatgacacctccgactgtgccaccta
caccttcagctcgggaatcaatgccattcaggagtggtataagctacact
acatgaacatcatggcacaggtgcctctggagaagaaaatcaacatgagc
tattctgctgaggagctgctggtgacctgcttctttgatggagtgtcctg
tgatgccaggaatttcacgcttttCcaccacccgatgcatgggaattgct
atactttcaacaacagagaaaatgagaccattctcagcacctccatgggg
ggcagcgaatatgggctgcaagtcattttgtacataaacgaagaggaata
caacccattcctcgtgtcctccactggagctaaggtgatcatccatcggc
aggatgagtatcccttcgtcgaagatgtgggaacagagattgagacagca
atggtcacctctataggaatgcacctgatctgcctCcattcatgcttcca
gacaaagatggtggagaaatgtgggtgtgcccagtacagccagcctctac
ctcctgcagccaactactgcaactaccagcagcaccccaactggatgtat
tgttactaccaactgcatcgagcctttgtccaggaagagctgggctgcca
gtctgtgtgcaaggaagcctgcagctttaaagagtggacactaaccacaa
gcctggcacaatggccatctgtggtttcggagaagtggttgctgcctgtt
ctcacttgggaccaaggccggcaagtaaacaaaaagctcaacaagacaga
cttgGccaaactcttgatattctacaaagacctgaaccagagatccatca
tggagagcccagccaacagtattgagatgcttctgtccaacttcggtggc
cagctgggcctgtggatgagctgctctgttgtctgcgtcatcgagatcat
cgaggtcttcttcattgacttcttctctatcattgcccgccgccagtggc
agaaagccaaggagtggtgggcctggaaacaggctcccccatgtccagaa
gctccccgtagcccacagggccaggacaatccagccctggatatagacga
tgacctacccactttcaactctgctttgcacctgcctccaGccctaggaa
cccaagtgcccggcacaccgccccccaaatacaataccttgcgcttggag
agggccttttccaaccagctcacagatacccagatgctGgatgagctctg
a

γA splice variant predicted protein sequence (SEQ ID NO:20):

MAPGEKIKAKIKKNLPVTGPQAPTIKELMRWYCLNTNTHGCRRIVVSRGR
LRRLLIGFTLTAVALILWQCALLVFSFYTVSVSIKVHFRKLDFPAVTICN
INPYKYSTVRHLLADLEQETREALKSLYGFPESRKRREAESWNSVSEGKQ
PRFSHRIPLLIFDQDEKGKARDFFTGRKRKVGGSIIHKASNVMHIESKQV
VGFQLCSNDTSDCATYTFSSGINAIQEWYKLHYMNIMAQVPLEKKINMSY
SAEELLVTCFFDGVSCDARNFTLFHHPMHGNCYTFNNRENETILSTSMGG
SEYGLQVILYINEEEYNPFLVSSTGAKVIIHRQDEYPFVEDVGTEIETAM
VTSIGMHLICLHSCFQTKMVEKCGCAQYSQPLPPAANYCNYQQHPNWMYC
YYQLHRAFVQEELGCQSVCKEACSFKEWTLTTSLAQWPSVVSEKWLLPVL
TWDQGRQVNKKLNKTDLAKLLIFYKDLNQRSIMESPANSIEMLLSNFGGQ
LGLWMSCSVVCVIEIIEVFFIDFFSIIARRQWQKAKEWWAWKQAPPCPEA
PRSPQGQDNPALDIDDDLPTFNSALHLPPALGTQVPGTPPPKYNTLRLER
AFSNQLTDTQMLDEL

In total, 5 ENaC variants were found (α1A, α2A, βA, βB, and γA) in the tissue analyzed herein that were also found in ILSbio tissue disclosed in our earlier patent application. One variant, β*, is a new variant that was not previously identified by Senomyx. The β* ENaC splice variant is especially interesting because it was observed in ˜5% of clones, which corresponds to the ˜10% taste cells contained in the UCSD CV taste tissue. In addition, the β* variant removes a small region of the β extracellular loop required for activation of kidney αβγ ENaC channels by our most-potent thio-indole enhancers, including 6363969. Lack of a kidney ENaC enhancer binding site on taste ENaC channels could account for the inability of identified kidney ENaC enhancers to promote human salt taste.

In conclusion, this invention identifies ENaC channel splice variant sequences expressed at the mRNA level in human taste tissue. These splice variants may be used to generate amiloride-insensitive channels that constitute the primary receptor for salt taste on the human tongue. Identification of enhancers of a taste ENaC channel would have significant use as salty taste enhancer additives to foods and beverages in order to retain the desired salty taste at reduced salt concentrations. Applications of these sequences and the clamed embodiments include the following:
1) Sequences can be used in identification of taste ENaC enhancers using Senomyx's oocyte assay enhancer functional screen.
2) Sequences can be used in identification of salt taste enhancers using Senomyx's oocyte assay enhancer functional screen in combination with human salt taste sensory tests.
3) Purified and isolated α1A splice variant nucleotide sequence contained in SEQ ID NO:3
4) A purified and isolated α2A splice variant nucleotide sequence (SEQ ID NO 7) is claimed
5) A purified and isolated βA splice variant nucleotide sequence (SEQ ID NO:11) is claimed
6) A purified and isolated βB splice variant nucleotide sequence (SEQ ID NO:13) is claimed
7) A purified and isolated β* splice variant nucleotide sequence (SEQ ID NO:15) is claimed
8) A purified and isolated γA splice variant nucleotide sequence (SEQ ID NO:19) is claimed
9) A purified and isolated α1A splice variant polypeptide sequence (SEQ ID NO:4) is claimed
10) A purified and isolated α2A splice variant polypeptide sequence (SEQ ID NO:8) is claimed
11) A purified and isolated βA splice variant polypeptide sequence (SEQ ID NO:12) is claimed
12) A purified and isolated βB splice variant polypeptide sequence (SEQ ID NO:14) is claimed
13) A purified and isolated β* splice variant polypeptide sequence (SEQ ID NO:16) is claimed
14) A purified and isolated γA splice variant polypeptide sequence (SEQ ID NO:20) is claimed

While the invention has been described by way of example embodiments, it is understood that the words which have been used herein are words of description, rather than words of limitation. Changes may be made, within the purview of the appealed claims, without departing from the scope and spirit of the invention in its broadest aspects. Although the invention has been described herein with reference to particular means, materials, and embodiments, it is understood that the invention is not limited to the particulars disclosed. The invention extends to all equivalent structures, means, and uses which are within the scope of the appended claims.

1. A purified and isolated nucleic acid sequence that encodes an ENaC α splice variant expressed in human taste tissue.

2. The purified and isolated nucleic acid sequence according to claim 1 that is contained in SEQ ID NO:3.

3. The purified and isolated nucleic acid sequence according to claim 1 that is contained in SEQ ID NO:7.

4. A purified and isolated nucleic acid sequence that encodes an ENaC β splice variant that is expressed in human taste tissue.

5. The purified and isolated nucleic acid sequence according to claim 4 that is contained SEQ ID NO:11.

6. The purified and isolated nucleic acid sequence according to claim 4 that is contained in SEQ ID NO:13.

7. The purified and isolated nucleic acid sequence according to claim 6 that is contained in SEQ ID NO:15.

8. A purified and isolated nucleic acid sequence that encodes an ENaC γ subunit that is expressed in human taste tissue.

9. The purified and isolated nucleic acid sequence according to claim 9 that is contained in SEQ ID NO:19.

10. A purified and isolated human ENaC subunit splice variant polypeptide that is comprised in an ENaC expressed in endogenous human taste tissue that is insensitive to amiloride.

11. The purified and isolated human ENaC subunit polypeptide according to claim 10 which is an α subunit polypeptide.

12. The purified and isolated human ENaC α subunit splice variant according to claim 11 which is selected from those contained in SEQ ID NO:4, and SEQ ID NO:6, SEQ ID NO:8.

13. The purified and isolated human ENaC subunit polypeptide according to claim 12 which a β subunit polypeptide.

14. The purified and isolated human ENaC β subunit polypeptide according to claim 13 which is selected from the group consisting of SEQ ID NO:12, SEQ ID NO:14 and SEQ ID NO:16.

15. The purified and isolated human ENaC subunit polypeptide according to claim 10 which is a subunit polypeptide.

16. The purified and isolated human ENaC γ subunit polypeptide according to claim 14 which is contained in SEQ ID NO:20.

17. A recombinant cell which expresses at least one splice variant nucleic acid sequence according claims 1.

18. The recombinant cell of claim 17 which is an amphibian or mammalian cell.

19. The recombinant cell of claim 17 which is a frog oocyte.

20. The recombinant cell of claim 18 which is selected from the group consisting of MDCK, HEK293, HEK293T, BHK, COS, N1H3T3, Swiss 3T3 and CHO cells.

21. A recombinant cell according to claim 17 which expresses an amiloride-insensitive ENaC.

22. The recombinant cell of claim 17 which is an amphibian oocyte.

23. The recombinant cell of claim 17 which is a mammalian oocyte.

24. A method of identifying a compound that modulates salty taste in humans comprising:
(i) contacting a cell that expresses an amiloride-insensitive ENaC containing at least one ENaC splice variant polypeptide expressed in human taste and tissue with at least one a putative taste modulatory compound;
(ii) determining whether said compound has a modulatory effect on ENaC function; and
(iii) identifying said compound as a putative modulatory of human salty taste if appreciably modulates said ENaC function.

25. The method of claim 24 wherein said compound is further tested in taste tests to confirm its modulatory effect (enhancing or inhibitory) on human salty taste.

26. The assay of claim 24 which comprises:
a mammalian cell-based high throughput assay for the profiling and screening of putative modulators of an epithelial sodium channel (ENaC) comprising:
contacting a said cell expressing alpha, beta and gamma subunits or delta, beta and gamma subunits or a variant, fragment or functional equivalent of each of these three subunits and preloaded with a membrane potential fluorescent dye or a sodium fluorescent dye with at least one putative modulator compound in the presence of sodium or lithium; and
monitoring anion mediated changes in fluorescence of the test cell in the presence of the putative modulator/ENaC interactions compared to changes in the absence of the modulator to determine the extent of ENaC modulation.

27. The assay of claim 26 wherein the anion is sodium or lithium.

28. The assay of claim 26 wherein said cells are seeded onto a wall of a multi-wall test plate.

29. The assay of claim 26 wherein said cells are loaded with a membrane potential dye that is responsive to changes in fluorescence.

30. The assay of claim 26 wherein fluorescence change is detected using a fluorescence plate reader or voltage imaging plate reader.

31. The assay of claim 26 wherein said cell expresses human ENaC α, β and γ subunits and at least two of said subunits comprise a splice variant expressed in human taste tissue.

32. The assay of claim 31 wherein all of said α, β and γ subunits are splice variants expressed in human taste tissue.

33. The assay according to claim 26 wherein said test cells are selected from the group consisting of MDCK, HEK293, HEK293T, BHK, COS, N1H3T3, Swiss 3T3 and CHO cells.


SEQUENCE LISTING (24 sequences)

SEQ ID NO: 1 - DNA, 2100 bp, Homo sapiens
atggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctgagagggaaggagaggtttctcacaccaaggcagatgctcctctggtgggagggtgctggccctggcaagattgaaggatgtgcaggaattc

SEQ ID NO: 2 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 3 - DNA, 1953 bp, Homo sapiens
atggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacgtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctga

SEQ ID NO: 4 - protein, 650 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 5 - DNA, 2187 bp, Homo sapiens
atgggcatggccaggggcagcctcactcgggttccaggggtgatgggagagggcactcagggcccagagctcagccttgaccctgacccttgctctccccaatccactccggggctcatgaaggggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacggtctgtccctgatgctgcgcgcagagcagaatgacttcattcccctgctgtccacagtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctga

SEQ ID NO: 6 - protein, 728 aa, Homo sapiens
MGMARGSLTRVPGVMGEGTQGPELSLDPDPCSPQSTPGLMKGNKLEEQDPRPLQPIPGLMEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 7 - DNA, 2130 bp, Homo sapiens
atgggcatggccaggggcagcctcactcgggttccaggggtgatgggagagggcactcagggcccagagctcagccttgaccctgacccttgctctccccaatccactccggggctcatgaaggggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtacccggaaattaaagaggagctggaggagctggaccgcatcacagagcagacgctctttgacctgtacaaatacagctccttcaccactctcgtggccggctcccgcagccgtcgcgacctgcgggggactctgccgcaccccttgcagcgcctgagggtcccgcccccgcctcacggggcccgtcgagcccgtagcgtggcctccagcttgcgggacaacaacccccaggtggactggaaggactggaagatcggcttccagctgtgcaaccagaacaaatcggactgcttctaccagacatactcatcaggggtggatgcggtgagggagtggtaccgcttccactacatcaacatcctgtcgaggctgccagagactctgccatccctggaggaggacacgctgggcaacttcatcttcgcctgccgcttcaaccaggtctcctgcaaccaggcgaattactctcacttccaccacccgatgtatggaaactgctatactttcaatgacaagaacaactccaacctctggatgtcttccatgcctggaatcaacaacgtgactggggcccgggtaatggtgcacgggcaggatgaacctgcctttatggatgatggtggctttaacttgcggcctggcgtggagacctccatcagcatgaggaaggaaaccctggacagacttgggggcgattatggcgactgcaccaagaatggcagtgatgttcctgttgagaacctttacccttcaaagtacacacagcaggtgtgtattcactcctgcttccaggagagcatgatcaaggagtgtggctgtgcctacatcttctatccgcggccccagaacgtggagtactgtgactacagaaagcacagttcctgggggtactgctactataagctccaggttgacttctcctcagaccacctgggctgtttcaccaagtgccggaagccatgcagcgtgaccagctaccagctctctgctggttactcacgatggccctcggtgacatcccaggaatgggtcttccagatgctatcgcgacagaacaattacaccgtcaacaacaagagaaatggagtggccaaagtcaacatcttcttcaaggagctgaactacaaaaccaattctgagtctccctctgtcacgatggtcaccctcctgtccaacctgggcagccagtggagcctgtggttcggctcctcggtgttgtctgtggtggagatggctgagctcgtctttgacctgctggtcatcatgttcctcatgctgctccgaaggttccgaagccgatactggtctccaggccgagggggcaggggtgctcaggaggtagcctccaccctggcatcctcccctccttcccacttctgcccccaccccatgtctctgtccttgtcccagccaggccctgctccctctccagccttgacagcccctccccctgcctatgccaccctgggcccccgcccatctccagggggctctgcaggggccagttcctccacctgtcctctgggggggccctga

SEQ ID NO: 8 - protein, 709 aa, Homo sapiens
MGMARGSLTRVPGVMGEGTQGPELSLDPDPCSPQSTPGLMKGNKLEEQDPRPLQPIPGLMEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 9 - DNA, 1923 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgtggacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 10 - protein, 640 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGVLVDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 11 - DNA, 1815 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgtggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 12 - protein, 604 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGVLVACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 13 - DNA, 1458 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgtggacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 14 - protein, 485 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKNFTSIFYPHYGNCYIFNWGMTEKALPSANPGTEFGLKLILDIGQEDYVPFLASTAGVRLMLHEQRSYPFIRDEGIYAMSGTETSIGVLVDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 15 - DNA, 1914 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 16 - protein, 637 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 17 - DNA, 1920 bp, Homo sapiens
atgcacgtgaagaagtacctgctgaagggcctgcatcggctgcagaagggccccggctacacgtacaaggagctgctggtgtggtactgcgacaacaccaacacccacggccccaagcgcatcatctgtgaggggcccaagaagaaagccatgtggttcctgctcaccctgctcttcgccgccctcgtctgctggcagtggggcatcttcatcaggacctacttgagctgggaggtcagcgtctccctctccgtaggcttcaagaccatggacttccccgccgtcaccatctgcaatgctagccccttcaagtattccaaaatcaagcatttgctgaaggacctggatgagctgatggaagctgtcctggagagaatcctggctcctgagctaagccatgccaatgccaccaggaacctgaacttctccatctggaaccacacacccctggtccttattgatgaacggaacccccaccaccccatggtccttgatctctttggagacaaccacaatggcttaacaagcagctcagcatcagaaaagatctgtaatgcccacgggtgcaaaatggccatgagactatgtagcctcaacaggacccagtgtaccttccggaacttcaccagtgctacccaggcattgacagagtggtacatcctgcaggccaccaacatctttgcacaggtgccacagcaggagctagtagagatgagctaccccggcgagcagatgatcctggcctgcctattcggagctgagccctgcaactaccggaacttcacgtccatcttctaccctcactatggcaactgttacatcttcaactggggcatgacagagaaggcacttccttcggccaaccctggaactgaattcggcctgaagttgatcctggacataggccaggaagactacgtccccttccttgcgtccacggccggggtcaggctgatgcttcacgagcagaggtcataccccttcatcagagatgagggcatctacgccatgtcggggacagagacgtccatcggggtactcgacaagcttcagcgcatgggggagccctacagcccgtgcaccgtgaatggttctgaggtccccgtccaaaacttctacagtgactacaacacgacctactccatccaggcctgtcttcgctcctgcttccaagaccacatgatccgtaactgcaactgtggccactacctgtacccactgccccgtggggagaaatactgcaacaaccgggacttcccagactgggcccattgctactcagatctacagatgagcgtggcgcagagagagacctgcattggcatgtgcaaggagtcctgcaatgacacccagtacaagatgaccatctccatggctgactggccttctgaggcctccgaggactggattttccacgtcttgtctcaggagcgggaccaaagcaccaatatcaccctgagcaggaagggaattgtcaagctcaacatctacttccaagaatttaactatcgcaccattgaagaatcagcagccaataacatcgtctggctgctctcgaatctgggtggccagtttggcttctggatggggggctctgtgctgtgcctcatcgagtttggggagatcatcatcgactttgtgtggatcaccatcatcaagctggtggccttggccaagagcctacggcagcggcgagcccaagccagctacgctggcccaccgcccaccgtggccgagctggtggaggcccacaccaactttggcttccagcctgacacggccccccgcagccccaacactgggccctaccccagtgagcaggccctgcccatcccaggcaccccgccccccaactatgactccctgcgtctgcagccgctggacgtcatcgagtctgacagtgagggtgatgccatctaa

SEQ ID NO: 18 - protein, 639 aa, Homo sapiens
MHVKKYLLKGLHRLQKGPGYTYKELLVWYCDNTNTHGPKRIICEGPKKKAMWFLLTLLFAALVCWQWGIFIRTYLSWEVSVSLSVGFKTMDFPAVTICNASPFKYSKIKHLLKDLDELMEAVLERILAPELSHANATRNLNFSIWNHTPLVLIDERNPHHPMVLDLFGDNHNGLTSSSASEKICNAHGCKMAMRLCSLNRTQCTFRNFTSATQALTEWYILQATNIFAQVPQQELVEMSYPGEQMILACLFGAEPCNYRNFTSIFYPHYGNCYIFNWGMTEKALPSANPGTEFGLKLILDIGQEDYVPFLASTAGVRLMLHEQRSYPFIRDEGIYAMSGTETSIGVLDKLQRMGEPYSPCTVNGSEVPVQNFYSDYNTTYSIQACLRSCFQDHMIRNCNCGHYLYPLPRGEKYCNNRDFPDWAHCYSDLQMSVAQRETCIGMCKESCNDTQYKMTISMADWPSEASEDWIFHVLSQERDQSTNITLSRKGIVKLNIYFQEFNYRTIEESAANNIVWLLSNLGGQFGFWMGGSVLCLIEFGEIIIDFVWITIIKLVALAKSLRQRRAQASYAGPPPTVAELVEAHTNFGFQPDTAPRSPNTGPYPSEQALPIPGTPPPNYDSLRLQPLDVIESDSEGDAI

SEQ ID NO: 19 - DNA, 1950 bp, Homo sapiens
atggcacccggagagaagatcaaagccaaaatcaagaagaatctgcccgtgacgggccctcaggcgccgaccattaaagagctgatgcggtggtactgcctcaacaccaacacccatggctgtcgccgcatcgtggtgtcccgcggccgtctgcgccgcctcctctggatcgggttcacactgactgccgtggccctcatcctctggcagtgcgccctcctcgtcttctccttctatactgtctcagtttccatcaaagtccacttccggaagctggattttcctgcagtcaccatctgcaacatcaacccctacaagtacagcaccgttcgccaccttctagctgacttggaacaggagaccagagaggccctgaagtccctgtatggctttccagagtcccggaagcgccgagaggcggagtcctggaactccgtctcagagggaaagcagcctagattctcccaccggattccgctgctgatctttgatcaggatgagaagggcaaggccagggacttcttcacagggaggaagcggaaagtcggcggtagcatcattcacaaggcttcaaatgtcatgcacatcgagtccaagcaagtggtgggattccaactgtgctcaaatgacacctccgactgtgccacctacaccttcagctcgggaatcaatgccattcaggagtggtataagctacactacatgaacatcatggcacaggtgcctctggagaagaaaatcaacatgagctattctgctgaggagctgctggtgacctgcttctttgatggagtgtcctgtgatgccaggaatttcacgcttttccaccacccgatgcatgggaattgctatactttcaacaacagagaaaatgagaccattctcagcacctccatggggggcagcgaatatgggctgcaagtcattttgtacataaacgaagaggaatacaacccattcctcgtgtcctccactggagctaaggtgatcatccatcggcaggatgagtatcccttcgtcgaagatgtgggaacagagattgagacagcaatggtcacctctataggaatgcacctgacagagtccttcaagctgagtgagccctacagtcagtgcacggaggacgggagtgacgtgccaatcaggaacatctacaacgctgcctactcgctccagatctgccttcattcatgcttccagacaaagatggtggagaaatgtgggtgtgcccagtacagccagcctctacctcctgcagccaactactgcaactaccagcagcaccccaactggatgtattgttactaccaactgcatcgagcctttgtccaggaagagctgggctgccagtctgtgtgcaaggaagcctgcagctttaaagagtggacactaaccacaagcctggcacaatggccatctgtggtttcggagaagtggttgctgcctgttctcacttgggaccaaggccggcaagtaaacaaaaagctcaacaagacagacttggccaaactcttgatattctacaaagacctgaaccagagatccatcatggagagcccagccaacagtattgagatgcttctgtccaacttcggtggccagctgggcctgtggatgagctgctctgttgtctgcgtcatcgagatcatcgaggtcttcttcattgacttcttctctatcattgcccgccgccagtggcagaaagccaaggagtggtgggcctggaaacaggctcccccatgtccagaagctccccgtagcccacagggccaggacaatccagccctggatatagacgatgacctacccactttcaactctgctttgcacctgcctccagccctaggaacccaagtgcccggcacaccgccccccaaatacaataccttgcgcttggagagggccttttccaaccagctcacagatacccagatgctagatgagctctga

SEQ ID NO: 20 - protein, 649 aa, Homo sapiens
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGSEYGLQVILYINEEEYNPFLVSSTGAKVIIHRQDEYPFVEDVGTEIETAMVTSIGMHLTESFKLSEPYSQCTEDGSDVPIRNIYNAAYSLQICLHSCFQTKMVEKCGCAQYSQPLPPAANYCNYQQHPNWMYCYYQLHRAFVQEELGCQSVCKEACSFKEWTLTTSLAQWPSVVSEKWLLPVLTWDQGRQVNKKLNKTDLAKLLIFYKDLNQRSIMESPANSIEMLLSNFGGQLGLWMSCSVVCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 21 - DNA, 1849 bp, Homo sapiens
atggcacccggagagaagatcaaagccaaaatcaagaagaatctgcccgtgacgggccctcaggcgccgaccattaaagagctgatgcggtggtactgcctcaacaccaacacccatggctgtcgccgcatcgtggtgtcccgcggccgtctgcgccgcctcctctggatcgggttcacactgactgccgtggccctcatcctctggcagtgcgccctcctcgtcttctccttctatactgtctcagtttccatcaaagtccacttccggaagctggattttcctgcagtcaccatcgcaacatcaacccctacaagtacagcaccgttcgccaccttctagctgacttggaacaggagaccagagaggccctgaagtccctgtatggctttccagagtcccggaagcgccgagaggcggagtcctggaactccgtctcagagggaaagcagcctagattctcccaccggattccgctgctgatctttgatcaggatgagaagggcaaggccagggacttcttcacgggaggaagcggaaagtcggcggtagcatcattcacaaggcttcaaatgtcatgcacatcgagtccaagcaagtggtgggattccaactgtgctcaaatgacacctccgactgtgccacctacaccttcagctcgggaatcaatgccattcaggagtggtataagctacactacatgaacatcatggcacaggtgcctctggagaagaaaatcaacatgagctattctgctgaggagctgctggtgacctgcttctttgatggagtgtcctgtgatgccaggaatttcacgcttttccaccacccgatgcatgggaattgctatactttcaacaacagagaaaatgagaccattctcagcacctccatggggggcagcgaatatgggctgcaagtcattttgtacataaacgaagaggaatacaacccattcctcgtgtcctccactggagctaaggtgatcatccatcggcaggatgagtatcccttcgtcgaagatgtgggaacagagattgagacagcaatggtcacctctataggaatgcacctgatctgcctccattcatgcttccagacaaagatggtggagaaatgtgggtgtgcccagtacagccagcctctacctcctgcagccaactactgcaactaccagcagcaccccaactggatgtattgttactaccaactgcatcgagcctttgtccaggaagagctgggctgccagtctgtgtgcaaggaagcctgcagctttaaagagtggacactaaccacaagcctggcacaatggccatctgtggtttcggagaagtggttgctgcctgttctcacttgggaccaaggccggcaagtaaacaaaaagctcaacaagacagacttggccaaactcttgatattctacaaagacctgaaccagagatccatcatggagagcccagccaacagtattgagatgcttctgtccaacttcggtggccagctgggcctgtggatgagctgctctgttgtctgcgtcatcgagatcatcgaggtcttcttcattgacttcttctctatcattgcccgccgccagtggcagaaagccaaggagtggtgggcctggaaacaggctcccccatgtccagaagctccccgtagcccacagggccaggacaatccagccctggatatagacgatgacctacccactttcaactctgctttgcacctgcctccagccctaggaacccaagtgcccggcacaccgccccccaaatacaataccttgcgcttggagagggccttttccaaccagctcacagatacccagatgctggatgagctctga

SEQ ID NO: 22 - protein, 617 aa, Homo sapiens
MAPGEKIKAKIKKNLPVTGPQAPTIKELMRWYCLNTNTHGCRRIVVSRGRLRRLLWIGFTLTAVALILWQCALLVFSFYTVSVSIKVHFRKLDFPAVTICNINPYKYSTVRHLLADLEQETREALKSLYGFPESRKRREAESWNSVSEGKQPRFSHRIPLLIFDQDEKGKARDFFTGRKRKVGGSIIHKASNVMHIESKQVVGFQLCSNDTSDCATYTFSSGINAIQEWYKLHYMNIMAQVPLEKKINMSYSAEELLVTCFFDGVSCDARNFTLFHHPMHGNCYTFNNRENETILSTSMGGEYSGLQVILYINNEEEYNPFLVSSTGAKVIIHRQDEYPFVEDVGTEIETAMVTSIGMHLICLHSCFQTKMVEKCGCAQYSQPLPPAANYCNYQQHPNWMYCYYQLHRAFVQEELGCQSVCKEACSFKEWTLTTSLAQWPSVVSEKWLLPVLTWDQGRQVNKKLNKTDLAKLLIFYKDLNQRSIMESPANSIEMLLSNFGGQLGLWMSCSVVCVIEIIEVFFIDFFSIIARRQWQKAKEWWAWKQAPPCPEAPRSPQGQDNPALDIDDDLPTFNSALHLPPALGTQVPGTPPPKYNTLRLERAFSNQLTDTQMLDEL

SEQ ID NO: 23 - DNA, 2597 bp, Homo sapiens
actcgggaaggccacacagccagtgacgaagctgtgattcacacaggcctgggtgactccagcatggctttcctctccaggacgtcaccggtggcagctgcttccttccagagccggcaggaggccagaggctccatcctgcttcagagctgccagctgcccccgcaatggctgagcaccgaagcatggacgggagaatggaagcagccacacgggggggctctcacctccagatcgcctgggcctgtggctccccagaggccctgccacctgaagggatggcagcacagacccactcagcacaacgctgcctgcaaacagggccaggctgcagcccagacgccccccaggccggggccaccatcagcaccaccaccaccacccaaggaggggcaccaggaggggctggtggagctgcccgcctcgttccgggagctgctcaccttcttctgcaccaatgccaccatccacggcgccatccgcctggtctgctcccgcgggaaccgcctcaagacgacgtcctgggggctgctgtccctgggagccctggtcgcgctctgctggcagctggggctcctctttgagcgtcactggcaccgcccggtcctcatggccgtctctgtgcactcggagcgcaagctgctcccgctggtcaccctgtgtgacgggaacccacgtcggccgagtccggtcctccgccatctggagctgctggacgagtttgccagggagaacattgactccctgtacaacgtcaacctcagcaaaggcagagccgccctctccgccactgtcccccgccacgagccccccttccacctggaccgggagatccgtctgcagaggctgagccactcgggcagccgggtcagagtggggttcagactgtgcaacagcacgggcggcgactgcttttaccgaggctacacgtcaggcgtggcggctgtccaggactggtaccacttccactatgtggatatcctggccctgctgcccgcggcatgggaggacagccacgggagccaggacggccacttcgtcctctcctgcagttacgatggcctggactgccaggcccgacagttccggaccttccaccaccccacctacggcagctgctacacggtcgatggcgtctggacagctcagcgccccggcatcacccacggagtcggcctggtcctcagggttgagcagcagcctcacctccctctgctgtccacgctggccggcatcagggtcatggttcacggccgtaaccacacgcccttcctggggcaccacagcttcagcgtccggccagggacggaggccaccatcagcatccgagaggacgaggtgcaccggctcgggagcccctacggccactgcaccgccggcggggaaggcgtggaggtggagctgctacacaacacctcctacaccaggcaggcctgcctggtgtcctgcttccagcagctgatggtggagacctgctcctgtggctactacctccaccctctgccggcgggggctgagtactgcagctctgcccggcaccctgcctggggacactgcttctaccgcctctaccaggacctggagacccaccggctcccctgtacctcccgctgccccaggccctgcagggagtctgcattcaagctctccactgggacctccaggtggccttccgccaagtcagctggatggactctggccacgctaggtgaacaggggctgccgcatcagagccacagacagaggagcagcctggccaaaatcaacatcgtctaccaggagctcaactaccgctcagtggaggaggcgcccgtgtactcggtgccgcagctgctctccgccatgggcagcctctacagcctgtggtttggggcctccgtcctctccctcctggagctcctggagctgctgctcgatgcttctgccctcaccctggtgctaggcggccgccggctccgcagggcgtggttctcctggcccagagccagccctgcctcaggggcgtccagcatcaagccagaggccagtcagatgcccccgcctgcaggcggcacgtcagatgacccggagcccagcgggcctcatctcccacgggtgatgcttccaggggttctggcgggagtctcagccgaagagagctgggctgggccccagccccttgagactctggacacctgaaccagacctgccagggctgtgcgatctcttggcctggtccttgcagctgtggcagcagcaggctccccagcggcccagggtgggccagaccagcagcccaggaagcagcacacgcggccgtggggaggcaggcaccgggcatgtcggcgcctctggtcaaaccacctacactgcctggggtgggtctcaaggaggcccggggcggaggggggttcccgcgtgcacacgagtgcggctggacgtgccgacacgcggtgatgtacccatgctccgtgtgtctgtgtctgcatgtccacacgtctgatgcacctgtgtacgtgtgtcaagcctagccacctcagctgcagggaggcagaaggcaaggcaggccccacggacacacttgggctgctctgaaataaagctgttgactccacctg

SEQ ID NO: 24 - protein, 704 aa, Homo sapiens
MAFLSRTSPVAAASFQSRQEARGSILLQSCQLPPQWLSTEAWTGEWKQPHGGALTSRSPGPVAPQRPCHLKGWQHRPTQHNAACKQGQAAAQTPPRPGPPSAPPPPPKEGHQEGLVELPASFRELLTFFCTNATIHGAIRLVCSRGNRLKTTSWGLLSLGALVALCWQLGLLFERHWHRPVLMAVSVHSERKLLPLVTLCDGNPRRPSPVLRHLELLDEFARENIDSLYNVNLSKGRAALSATVPRHEPPFHLDREIRLQRLSHSGSRVRVGFRLCNSTGGDCFYRGYTSGVAAVQDWYHFHYVDILALLPAAWEDSHGSQDGHFVLSCSYDGLDCQARQFRTFHHPTYGSCYTVDGVWTAQRPGITHGVGLVLRVEQQPHLPLLSTLAGIRVMVHGRNHTPFLGHHSFSVRPGTEATISIREDEVHRLGSPYGHCTAGGEGVEVELLHNTSYTRQACLVSCFQQLMVETCSCGYYLHPLPAGAEYCSSARHPAWGHCFYRLYQDLETHRLPCTSRCPRPCRESAFKLSTGTSRWPSAKSAGWTLATLGEQGLPHQSHRQRSSLAKINIVYQELNYRSVEEAPVYSVPQLLSAMGSLYSLWFGASVLSLLELLELLLDASALTLVLGGRRLRRAWFSWPRASPASGASSIKPEASQMPPPAGGTSDDPEPSGPHLPRVMLPGVLAGVSAEESWAGPQPLETLDT